(19) United States
(12) Patent Application Publication
(10) Pub. No.: US 2017/0281647 A1
(43) Pub. Date: Oct. 5, 2017

(54) STEROID HORMONE COMPOSITIONS IN MEDIUM CHAIN OILS

(71) Applicant: TherapeuticsMD, Inc., Boca Raton, FL (US)

(72) Inventors: ANNETTE SHADIACK, Somerset, NJ (US); PETER H.R. PERSICANER, Boca Raton, FL (US); PHILIP B. INSKEEP, East Lyme, CT (US); THORSTEINN THORSTEINSSON, Boynton Beach, FL (US); FREDERICK D. SANCILIO, Palm Beach Gardens, FL (US); JASON D. LEGASSIE, Riviera Beach, FL (US)

(21) Appl. No.: 15/475,068

(22) Filed: Mar. 30, 2017

Related U.S. Application Data
(60) Provisional application No. 62/317,402, filed on Apr. 1, 2016.

(51) Int. Cl. A61K 31/57; A61K 31/355; A61K 9/107; A61K 47/26; A61K 9/00; A61K 31/565; A61K 47/44

(52) U.S. Cl. CPC A61K 31/57; A61K 31/565; A61K 31/355; A61K 47/44; A61K 47/26; A61K 9/0053; A61K 9/107

(57) ABSTRACT

This disclosure provides a novel pharmaceutical composition capable of forming micelles upon exposure to the gastric environment. The pharmaceutical composition is suitable for delivering steroid hormones, and the combination of estradiol and progesterone in particular, to a subject in need thereof.

### CROSS-REFERENCES TO RELATED APPLICATIONS

This application claims priority to U.S. Provisional Patent Application No. 62/317,402, filed on Apr. 1, 2016, which application is incorporated herein by reference in its entirety.

### FIELD

This disclosure relates to the field of steroid hormones and in particular, provides a pharmaceutical composition comprising a fully-solubilized steroid hormone, such as estradiol and progesterone, having enhanced oral bioavailability compared with currently marketed pharmaceutical compositions.

### BACKGROUND

Steroid hormones are vital constituents for the proper functioning of the human body and can be classified into five groups based on the receptors to which they bind, namely: glucocorticoids, mineralocorticoids, androgens, estrogens, and progestogens. It is known that steroid hormones aid in regulating metabolism, regulating water and salt function, regulating immune function, controlling inflammation, and developing sexual characteristics.

Despite their wide ranging biological activity, steroid hormones are difficult to deliver to a subject experiencing a disease or disorder where additional steroid hormone could help treat the disease or disorder. Hormone Replacement Therapy (HRT) is an example of a medical treatment that is designed to increase hormone levels in women who lack adequate hormone production in order to treat conditions and diseases associated with low estrogen levels and/or low progesterone levels. HRT can mitigate and prevent symptoms caused by diminished circulating estrogen and progesterone hormones in a pre-menopausal, peri-menopausal, menopausal, or post-menopausal subject. However, progesterone and estradiol have extremely poor oral bioavailability due to their respective limited water solubility. As a result (and particularly with progesterone), when given orally they must be administered in a sufficiently high dose to obtain the desired pharmacokinetic profile. Higher dosages of progesterone, however, make co-formulating progesterone with estradiol very challenging, and higher dosages of progesterone are inherently less desirable as the greater the quantity dosed, the greater the likelihood that additional drug, beyond what the patient requires, could enter the body and exert an effect.

Progesterone (CAS#57-83-0), also known as P4 (pregn-4-ene-3,20-dione), is a C-21 steroid hormone involved in the female menstrual cycle, pregnancy, and embryogenesis of humans and other species. Progesterone belongs to a class of hormones called progestogens, and is the major naturally occurring, endogenous human progestogen. The use of progesterone and its analogues has many medical applications, both to address acute conditions as well as the long-term decline of natural progesterone levels. Undesirable side effects exist due to irregular, inconsistent, or decreased hormone production in pre-, peri-, menopausal, and post-menopausal females. Progesterone is indicated for use in the prevention of endometrial hyperplasia in non-hysterectomized postmenopausal women who are receiving systemic estrogen. Progesterone is also indicated for use in secondary amenorrhea.

Estradiol (CAS#50-28-2), also known as 17β-estradiol, oestradiol, or E2, is found endogenously in the human body and is the primary female sex hormone. Estradiol contributes to regulation of estrous and menstrual reproduction cycles in females, development of reproductive tissues, and maintenance of bone tissue, among other processes. Estradiol deficiency in female subjects is implicated in conditions such as preterm birth, sleep disturbances, mood changes, vulvo-vaginal atrophy, and osteoporosis.

Existing oral compositions are formulated such that high dosages of hormones or various synthetic estrogen and progesterone analogs are administered, and most oral progesterone compositions suffer from progesterone's limited absorption and bioavailability. Therefore, new oral compositions for more effective delivery of estradiol and progesterone are needed. The invention disclosed herein meets this and other needs.

### SUMMARY

This disclosure is directed to pharmaceutical compositions capable of fully solubilizing a steroid hormone, such as estradiol and/or progesterone, and that form micelles upon administration. The pharmaceutical compositions comprise estradiol and progesterone, various medium chain oils, a polysorbate, and, optionally, a terpene. In certain embodiments, the medium chain oils are predominantly C6-C14 medium chain oils. In certain embodiments, the optional terpene can be a monocyclic terpene such as d-limonene. Importantly, the pharmaceutical compositions disclosed herein can be used to deliver bio-identical estrogen, such as estradiol, and bio-identical progestogen, such as progesterone. The pharmaceutical compositions provide oral bioavailability, as measured by AUC, of the steroid hormone, which in certain embodiments is either or both estradiol and progesterone, of at least 125%, at least 150%, at least 175%, and in certain instances, at least 200% relative to a reference product such as ESTRACE for estradiol and PROMETRIUM for progesterone.

This disclosure further provides methods of treating, inhibiting, or preventing a condition or disorder characterized by a steroid hormone deficiency, and in particular, conditions or disorders characterized by low levels of estrogen and/or progesterone. The methods comprise administering to a subject a therapeutically effective amount of at least one pharmaceutical composition described herein.

In one embodiment, the present disclosure provides a pharmaceutical composition for providing enhanced oral bioavailability of estradiol and/or progesterone, the pharmaceutical composition comprising: estradiol, progesterone, a polysorbate, and a medium chain oil, wherein the medium chain oil comprises at least about 50 weight percent of a predominantly medium chain monoglyceride; and the progesterone is fully solubilized.

In certain embodiments, the medium chain oil comprises at least about 55 weight percent of a predominantly medium chain monoglyceride.

In certain embodiments, the medium chain oil comprises at least about 60 weight percent of a predominantly medium chain monoglyceride.

In certain embodiments, the medium chain oil comprises at least about 65 weight percent of a predominantly medium chain monoglyceride.

In certain embodiments, the medium chain oil comprises at least about 70 weight percent of a predominantly medium chain monoglyceride.

In certain embodiments, the polysorbate is selected from the group consisting of polysorbate 20, polysorbate 40, polysorbate 60, polysorbate 65, and polysorbate 80.

In certain embodiments, the pharmaceutical composition provides an oral bioavailability of at least about 150 percent as measured by an increase in AUC when compared to a reference product.

In certain embodiments, the pharmaceutical composition further comprises a polyoxyethylene hydrogenated castor oil.

In certain embodiments, the pharmaceutical composition further comprises a d-α-tocopherol polyethylene glycol succinate derivative.

In certain embodiments, the pharmaceutical composition further comprises a terpene.

In certain embodiments, the predominantly medium chain monoglyceride and the progesterone can be present at a weight ratio of about 10:1 to about 15:1.

In certain embodiments, the predominantly medium chain monoglyceride comprises at least about 60 weight percent of the medium chain oil.

In certain embodiments, the predominantly medium chain monoglyceride comprises at least about 70 weight percent of the medium chain oil.

In certain embodiments, the predominantly medium chain monoglyceride comprises at least about 80 weight percent of the medium chain oil.

In certain embodiments, the predominantly medium chain monoglyceride comprises at least about 90 weight percent of the medium chain oil.

In certain embodiments, the polysorbate is selected from the group consisting of polysorbate 20, polysorbate 40, polysorbate 60, polysorbate 65, and polysorbate 80.

In certain embodiments, the pharmaceutical composition provides an oral bioavailability of at least about 150 percent as measured by an increase in AUC when compared to a reference product.

In certain embodiments, the pharmaceutical composition further comprises a polyoxyethylene hydrogenated castor oil.

In certain embodiments, the pharmaceutical composition further comprises a d-α-tocopherol polyethylene glycol succinate derivative.

In certain embodiments, the pharmaceutical composition further comprises a terpene.

In certain embodiments, the present disclosure provides an oral, fully-solubilized estradiol and progesterone pharmaceutical composition comprising: estradiol, progesterone, and a polysorbate in a weight ratio of from about 1:2 to about 2:1 ((estradiol plus progesterone):polysorbate); and a medium chain oil comprising a mixture of medium chain mono- and diglycerides, the medium chain oil not containing more than about 10 weight percent triglycerides.

In certain embodiments, the polysorbate is selected from the group consisting of polysorbate 20, polysorbate 40, polysorbate 60, polysorbate 65, and polysorbate 80.

In certain embodiments, the pharmaceutical composition provides an oral bioavailability of at least about 150 percent as measured by an increase in AUC when compared to a reference product.

In certain embodiments, the pharmaceutical composition further comprises a polyoxyethylene hydrogenated castor oil.

In certain embodiments, the pharmaceutical composition further comprises a d-α-tocopherol polyethylene glycol succinate derivative.

In certain embodiments, the pharmaceutical composition further comprises a terpene.

In other embodiments, the present disclosure provides a method of treating a disease or condition associated with reduced estrogen levels and/or reduced progesterone levels, the method comprising administering to a subject in need thereof a pharmaceutical composition comprising: estradiol, progesterone, a polysorbate, and a medium chain oil, wherein the medium chain oil comprises at least about 50 weight percent of a predominantly medium chain monoglyceride; and the progesterone is fully solubilized.

In certain embodiments, the method comprising administering to a subject in need thereof a pharmaceutical composition comprising: estradiol, progesterone, and a polysorbate in a weight ratio of from about 1:2 to about 2:1 ((estradiol plus progesterone):polysorbate); and a medium chain oil comprising a mixture of medium chain mono- and diglycerides, the medium chain oil not containing more than about 10 weight percent triglycerides.

In certain embodiments, the disease or condition associated with reduced progesterone levels is selected from the group consisting of endometrial hyperplasia; secondary amenorrhea; prevention of preterm birth; and osteoporosis.

In certain embodiments, the disease or condition associated with reduced progesterone levels is menopause.

In certain embodiments, the disease or condition associated with reduced estrogen levels is selected from the group consisting of symptoms associated with menopause, such as hot flushes/flashes, night sweats, vaginal dryness, urinary tract infections and loss of libido as well as other conditions and diseases such as heart disease and osteoporosis.

In certain embodiments, the disease or condition associated with reduced estrogen and/or progesterone levels is menopause. In certain embodiments, the disease or condition associated with reduced estrogen and/or progesterone levels is vasomotor symptoms of menopause, such as hot flushes/flashes, night sweats.

In addition to the foregoing, the present disclosure also provides a pharmaceutical composition for providing enhanced oral bioavailability of estradiol and/or progesterone, the pharmaceutical composition comprising: estradiol, progesterone, a polysorbate, and a medium chain oil, wherein the medium chain oil comprises at least about 50 weight percent of a first medium chain oil component; and the estradiol and the progesterone are both fully solubilized.

In some embodiments, the medium chain oil further comprises a second medium chain oil component.

In some embodiments, the first medium chain oil component is a predominantly medium chain monoglyceride.

In some embodiments, the second medium chain oil component is a predominantly medium chain diglyceride.

In some embodiments, the predominantly medium chain diglyceride is a mixed or complex diglyceride.

### BRIEF DESCRIPTION OF THE DRAWINGS/FIGURES

The foregoing summary, as well as the following detailed description, will be better understood when read in conjunction with the appended figures. For the purpose of illustration, the figures may describe the use of specific embodiments. It should be understood, however, that this disclosure is not limited to the precise embodiments discussed or described in these figures.

FIG. 1 is a graph of plasma concentration of progesterone vs. time for rats dosed with 20 μl of each of the various embodiments of the pharmaceutical composition described herein or 20 μl PROMETRIUM. Because of the way it is formulated, PROMETRIUM contains 400 mg progesterone/g of formulation. As such, the amount of progesterone dosed in rats treated with 20 μl PROMETRIUM far exceeded the amount of progesterone delivered to rats treated with 20 μl of the pharmaceutical compositions of this disclosure.

FIG. 2 is the log-linear version of FIG. 1.

FIG. 3 is a graph of plasma concentration of progesterone metabolite allopregnanolone sulfate vs. time for various embodiments of the pharmaceutical composition described herein and PROMETRIUM. As discussed above, the amount of progesterone administered to rats treated with 20 μl PROMETRIUM far exceeded the amount of progesterone administered to rats treated with 20 μl of the pharmaceutical compositions of this disclosure.

FIG. 4 is a log-linear version of FIG. 3.

FIG. 5 is a graph of plasma concentration of progesterone metabolite 20α-dihydroprogesterone vs. time for various embodiments of the pharmaceutical composition described herein and PROMETRIUM. As discussed above, the amount of progesterone administered to rats treated with 20 μl PROMETRIUM far exceeded the amount of progesterone administered to rats treated with 20 μl of the pharmaceutical compositions of this disclosure.

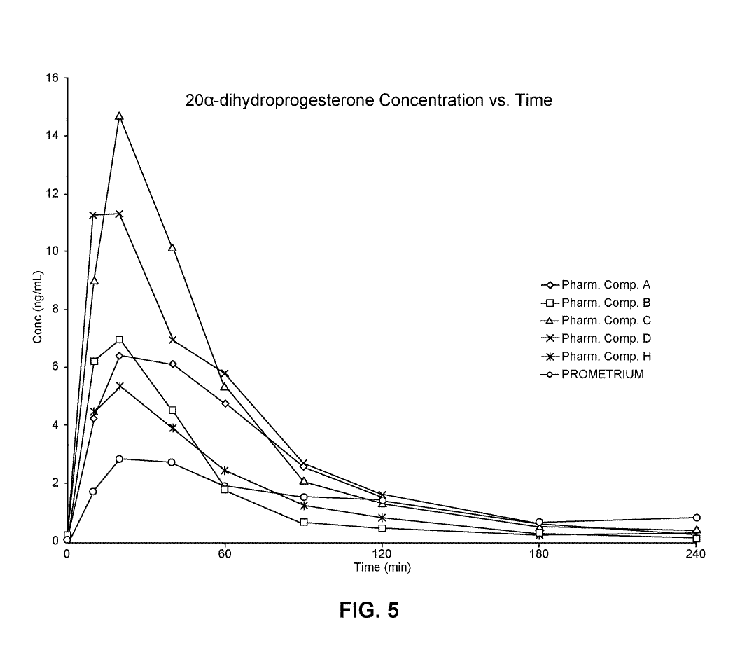

FIG. 6 is a log-linear version of FIG. 5.

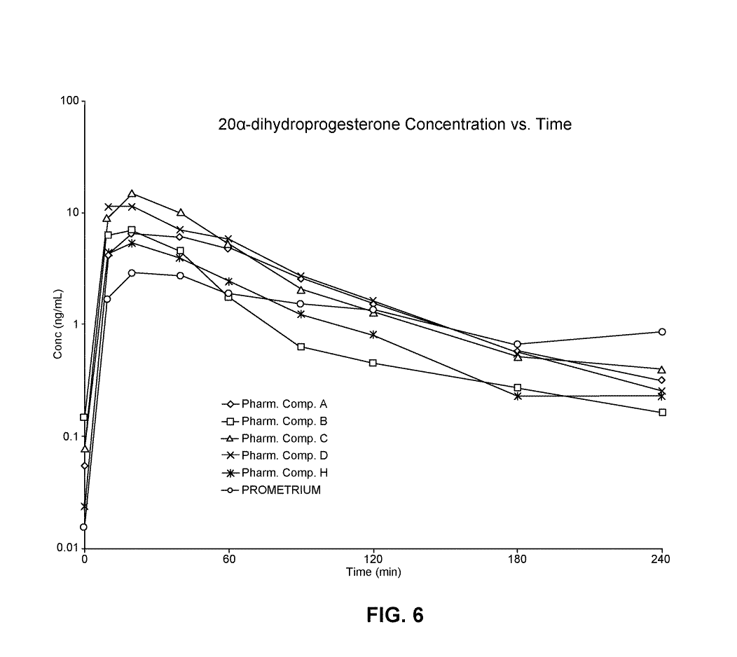

FIG. 7 is a graph of the plasma concentration of progesterone vs. time for fed and fasted rats dosed with 20 μl (25 mg/kg sample) of PROMETRIUM.

FIG. 8 is a graph of the plasma concentration of progesterone vs. time for fed and fasted rats dosed with 20 μl (3.7 mg/kg progesterone) of test pharmaceutical composition D in a gavage micro capsule.

FIG. 9 is a graph of the plasma concentration of progesterone vs. time for fed and fasted rats dosed with 20 μl (3.7 mg/kg progesterone) of test pharmaceutical composition C in a gavage micro capsule.

FIG. 10 is a graph of the plasma concentration of progesterone vs. time for fed and fasted rats dosed with 20 μl (3.7 mg/kg progesterone) of test pharmaceutical composition A in a gavage micro capsule.

FIG. 11 is graph of plasma concentration of progesterone vs. time for PROMETRIUM and test pharmaceutical composition D.

### DETAILED DESCRIPTION

### Definitions

The singular forms “a,” “an,” and “the” include plural referents unless the context clearly dictates otherwise.

As used herein, the term “or” is a logical disjunction (i.e., and/or) and does not indicate an exclusive disjunction unless expressly indicated as such with the terms “either,” “unless,” “alternatively,” and words of similar effect.

The term “area under the curve” (“AUC”) refers to the area under the curve defined by changes in the blood concentration of an active pharmaceutical ingredient (e.g., estradiol or progesterone), or a metabolite of the active pharmaceutical ingredient, over time following the administration of a dose of the active pharmaceutical ingredient. “AUC₀₋₂₈” is the area under the concentration-time curve extrapolated to infinity following the administration of a dose. “AUC₀₋ₜ” is the area under the concentration-time curve from time zero to time t following the administration of a dose, wherein t is the last time point with a measurable concentration.

The term “Cₘₐₓ” refers to the maximum value of blood concentration shown on the curve that represents changes in blood concentrations of an active pharmaceutical ingredient (e.g., progesterone or estradiol), or a metabolite of the active pharmaceutical ingredient, over time.

The term “tₘₐₓ” refers to the earliest time at which the blood concentration of an active pharmaceutical ingredient (e.g., estradiol or progesterone), or a metabolite of the active pharmaceutical ingredient is at its maximum value.

The term “bioavailability,” which has the meaning defined in 21 C.F.R. §320.1(a), refers to the rate and extent to which an active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action. For example, bioavailability can be measured as the amount of active ingredient in the blood (serum or plasma) as a function of time. Pharmacokinetic (PK) parameters such as AUC, Cₘₐₓ, or tₘₐₓ may be used to measure and assess bioavailability.

The term “bioequivalent,” has the meaning defined in 21 C.F.R. §320.1(e) and refers to the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In practice, two products are considered bioequivalent if the 90% confidence interval of the AUC or Cₘₐₓ is within 80.00% to 125.00%.

The term “bio-identical hormone” refers to an active pharmaceutical ingredient that is structurally identical to a hormone naturally or endogenously found in the human body (e.g., estradiol or progesterone).

As used herein, the term “about” refers to ±10% of the noted value, unless otherwise specified, and unless the upper bound of the range would exceed 100% of the pharmaceutical composition, in which case the upper limit of the range is limited to 99.9%. Thus, and by way of example only, a pharmaceutical composition including about 10 weight percent of a given compound could have from 9 to 11 weight percent of the compound. Similarly, a pharmaceutical composition including about 95 weight percent of a given compound could have from 85.5 to 99.9 weight percent of the compound in the pharmaceutical composition.

The term “estradiol” refers to (17β)-estra-1,3,5(10)-triene-3,17-diol. Estradiol is also interchangeably called 17β-estradiol, oestradiol, or E2, and is found endogenously in the human body. As used herein, estradiol refers to the bio-identical or body-identical form of estradiol found in the human body having the structure:

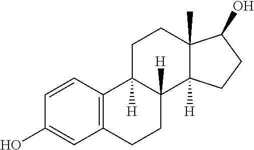

Estradiol is supplied in an anhydrous or hemi-hydrate form. For the purposes of this disclosure, the anhydrous form or the hemihydrate form can be substituted for the other by accounting for the water or lack of water according to well-known and understood techniques.

The term “solubilized estradiol” means that the estradiol or a portion thereof is solubilized or dissolved in the solubilizing agent(s) or the formulations disclosed herein. Solubilized estradiol may include estradiol that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the estradiol is “fully solubilized” with all or substantially all of the estradiol being solubilized or dissolved in the solubilizing agent. Fully solubilized estradiol may include estradiol that is about 97% solubilized, about 98% solubilized, about 99% solubilized or about 100% solubilized. Solubility can be expressed as a mass fraction (% w/w, which is also referred to as weight percent (wt %)).

As used herein, the term “progesterone” refers to pregn-4-ene-3,20-dione. As used herein, progesterone refers to the bioidentical or body-identical form of progesterone found in the human body having the structure:

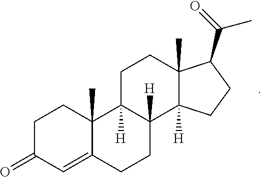

The term “solubilized progesterone” means that the progesterone or a portion thereof is solubilized or dissolved in the formulations disclosed herein. Solubilized progesterone may include progesterone that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the progesterone is “fully solubilized” with all or substantially all of the progesterone being solubilized or dissolved in the formulation. Fully solubilized progesterone may include progesterone that is about 97% solubilized, about 98% solubilized, about 99% solubilized or about 100% solubilized. Solubility can be expressed as a mass fraction (% w/w, which is also referred to as weight percent (wt %)).

The terms “micronized progesterone” and “micronized estradiol,” as used herein, include micronized progesterone and micronized estradiol having an X50 particle size value below about 15 microns or having an X90 particle size value below about 25 microns. The term “X50” means that one-half of the particles in a sample are smaller in diameter than a given number. For example, micronized progesterone having an X50 of 5 microns means that, for a given sample of micronized progesterone, one-half of the particles have a diameter of less than 5 microns. Similarly, the term “X90” means that ninety percent (90%) of the particles in a sample are smaller in diameter than a given number.

The solubility of a given steroid hormone can be measured using standard techniques by weighing a piece of filter paper, placing the weighed filter paper in a buchner funnel (porcelain or glass with a glass frit), and drawing a known quantity of pharmaceutical composition through the filter paper using vacuum (such as with a side-arm flask fitted with a neoprene collar). After drying for an appropriate period of time (either at room temperature or at elevated temperature), the filter paper is reweighed. The amount of steroid hormone on the filter paper is calculated and the amount of solubilized and insoluble steroid hormone is calculated.

The term “glyceride” refers to an ester of glycerol (1,2,3-propanetriol) with acyl radicals of fatty acids and is also known as an acylglycerol. If only one position of the glycerol molecule is esterified with a fatty acid, a “monoglyceride” or “monoacylglycerol” is produced; if two positions are esterified, a “diglyceride” or “diacylglycerol” is produced; and if all three positions of the glycerol are esterified with fatty acids, a “triglyceride” or “triacylglycerol” is produced. A glyceride is “simple” if all esterified positions contain the same fatty acid; whereas a glyceride is “mixed” if the esterified positions are substituted with different fatty acids. A glyceride is “complex” if it contains a combination of simple and mixed glycerides. The carbons of the glycerol backbone are designated sn-1, sn-2 and sn-3, with sn-2 being the middle carbon and sn-1 and sn-3 being the end carbons of the glycerol backbone.

As used herein, the term “hormone deficiency” refers to a low level of one or more steroid hormones in a subject. Normal hormone levels will vary from subject to subject and can be determined via known methods. Low hormone levels may or may not be associated with symptoms including, but not limited to, fatigue, irregular bleeding, lowered libido, and depression. Conditions that can be treated with estrogen and progesterone therapy to address estrogen and progesterone deficiencies include menopause-related symptoms including vasomotor symptoms (e.g., hot flashes and night sweats). Other hypoestrogenism related conditions and symptoms can also be treated with estrogen and progesterone therapy, including, for example and without limitation, vasomotor symptoms, sleep disturbances, mood changes, and vulvo-vaginal atrophy; and osteoporosis and other non-menopausal disease states or conditions that can be treated with supplemental estradiol and progesterone.

As used herein, the terms “host,” “subject,” and “patient” refer to any animal, including humans.

As used herein, the term “prevent” refers to the prophylactic treatment of a subject who is at risk of developing a condition (e.g., steroid hormone deficiency) resulting in a decrease in the probability that the subject will develop the condition.

The term “solubilizing agent” refers to an agent or combination of agents that solubilize an active pharmaceutical ingredient (e.g., estradiol or progesterone). For example and without limitation, suitable solubilizing agents include medium chain oils and other solvents and co-solvents that solubilize or dissolve an active pharmaceutical ingredient to a desirable extent. Solubilizing agents suitable for use in the pharmaceutical compositions disclosed herein are pharmaceutical grade solubilizing agents (e.g., pharmaceutical grade medium chain oils). It will be understood by those of skill in the art that other excipients or components can be added to or mixed with the solubilizing agent to enhance the properties or performance of the solubilizing agent or resulting pharmaceutical composition. Examples of such excipients include, but are not limited to, surfactants, emulsifiers, thickeners, colorants, flavoring agents, terpenes, etc. In some embodiments, the solubilizing agent is a medium chain oil and, in some other embodiments, the medium chain oil is combined with a co-solvent(s) or other excipient(s).

The term “medium chain” is used to describe the aliphatic chain length of fatty acid containing molecules. “Medium chain” specifically refers to fatty acids, fatty acid esters, or fatty acid derivatives that contain fatty acid aliphatic tails or carbon chains that contain, for example, 6 to 14 carbon atoms, 8 to 12 carbon atoms, or 8 to 10 carbon atoms.

The terms “medium chain fatty acid” and “medium chain fatty acid derivative” are used to describe fatty acids or fatty acid derivatives with aliphatic tails (i.e., carbon chains) having 6 to 14 carbon atoms. Fatty acids consist of an unbranched or branched aliphatic tail attached to a carboxylic acid functional group. Fatty acid derivatives include, for example, fatty acid esters and fatty acid containing molecules, including, without limitation, mono-, di- and triglycerides that include components derived from fatty acids. Fatty acid derivatives also include fatty acid esters of ethylene or propylene glycol. The aliphatic tails can be saturated or unsaturated (i.e., the latter having one or more double bonds between carbon atoms). In some embodiments, the aliphatic tails are saturated (i.e., no double bonds between carbon atoms). Medium chain fatty acids or medium chain fatty acid derivatives include those with aliphatic tails having 6-14 carbons, including those that are C6-C14, C6-C12, C8-C14, C8-C12, C6-C10, C8-C10, or others. Examples of medium chain fatty acids include, without limitation, caproic acid, caprylic acid, capric acid, lauric acid, myristic acid, and derivatives thereof. In certain embodiments, the medium chain fatty acids used to prepare the various medium chain oils described herein are C8, C10, or a combination thereof.

The term “oil,” as used herein, refers to any pharmaceutically acceptable oil, especially medium chain oils, and specifically excluding peanut oil, that can suspend or solubilize bioidentical progesterone or estradiol, including starting materials or precursors thereof, including micronized progesterone and/or micronized estradiol as described herein.

The terms “treat,” “treating,” “treatment,” and the like refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subjective parameters, including the results of a physical examination, neuropsychiatric examinations, or psychiatric evaluation.

The term “medium chain oil” refers to an oil wherein the composition of the fatty acid fraction of the oil is substantially medium chain (i.e., C6 to C14) fatty acids, i.e., the composition profile of fatty acids in the oil is substantially medium chain. As used herein, “substantially” means that between 20% and 100% (inclusive of the upper and lower limits) of the fatty acid fraction of the oil is made up of medium chain fatty acids, i.e., fatty acids with aliphatic tails (i.e., carbon chains) having 6 to 14 carbons. In some embodiments, about 25%, about 30%, about 35%, about 40%, about 45%, about 50%, about 55%, about 60%, about 65%, about 70%, about 75%, about 85%, about 90% or about 95% of the fatty acid fraction of the oil is made up of medium chain fatty acids. Those of skill in the art will readily appreciate that the terms “alkyl content” or “alkyl distribution” of an oil can be used in place of the term “fatty acid fraction” of an oil in characterizing a given oil or solubilizing agent, and these terms are used interchangeably herein. As such, medium chain oils suitable for use in the pharmaceutical compositions disclosed herein include medium chain oils wherein the fatty acid fraction of the oil is substantially medium chain fatty acids, or medium chain oils wherein the alkyl content or alkyl distribution of the oil is substantially medium chain alkyls, e.g., C6-C14 alkyls, but also including, for example, C6-C12 alkyls, C8-C12 alkyls, and C8-C10 alkyls. It will be understood by those of skill in the art that the medium chain oils suitable for use in the pharmaceutical compositions disclosed herein are pharmaceutical grade (e.g., pharmaceutical grade medium chain oils). Examples of medium chain oils include, for example and without limitation, medium chain fatty acids, medium chain fatty acid esters of glycerol (e.g., for example, mono-, di-, and triglycerides), medium chain fatty acid esters of propylene glycol, medium chain fatty acid derivatives of polyethylene glycol, and combinations thereof.

The term “ECN” or “equivalent carbon number” means the sum of the number of carbon atoms in the fatty acid chains of an oil, and can be used to characterize an oil as, for example, a medium chain oil or a long-chain oil. For example, tripalmitin (tripalmitic glycerol), which is a simple triglyceride containing three fatty acid chains of 16 carbon atoms, has an ECN of 3×16=48. Conversely, a triglyceride with an ECN=40 may have “mixed” fatty acid chain lengths of 8, 16, and 16; 10, 14, and 16; 8, 14, and 18; etc. Naturally occurring oils are frequently “mixed” with respect to specific fatty acids, but tend not to contain both long chain fatty acids and medium chain fatty acids in the same glycerol backbone. Thus, triglycerides with ECN's of 21-42 typically contain predominately medium chain fatty acids; while triglycerides with ECN's of greater than 43 typically contain predominantly long chain fatty acids. For example, the ECN of corn oil triglyceride in the USP would be in the range of 51-54. Medium chain diglycerides with ECN's of 12-28 will often contain predominately medium chain fatty chains, while diglycerides with ECN's of 32 or greater will typically contain predominately long chain fatty acid tails. Monoglycerides will have an ECN that matches the chain length of the sole fatty acid chain. Thus, monoglyceride ECN's in the range of 6-14 contain mainly medium chain fatty acids, and monoglycerides with ECN's 16 or greater will contain mainly long chain fatty acids.

The average ECN of a medium chain triglyceride oil is typically 21-42. For example, as listed in the US Pharmacopeia (USP), medium chain triglycerides have the following composition as the exemplary oil set forth in the table below:

| Fatty-acid Tail Length | % of oil | Exemplary Oil |
|---|---|---|
| 6 | ≦2.0 | 2.0 |
| 8 | 50.0-80.0 | 70.0 |
| 10 | 20.0-50.0 | 25.0 |
| 12 | ≦3.0 | 2.0 |
| 14 | ≦1.0 | 1.0 |

and would have an average ECN of 3*[(6*0.02)+(8*0.70)+(10*0.25)+(12*0.02)+(14*0.01)]=25.8. The ECN of the exemplary medium chain triglycerides oil can also be expressed as a range (per the ranges set forth in the USP) of 24.9-27.0. For oils that have mixed mono-, di-, and triglycerides, or single and double fatty acid glycols, the ECN of the entire oil can be determined by calculating the ECN of each individual component (e.g., C8 monoglycerides, C8 diglycerides, C10 monoglycerides, and C10 diglycerides) and taking the sum of the relative percentage of the component multiplied by the ECN normalized to a monoglyceride for each component. For example, an oil having C8 and C10 mono- and diglycerides shown in the table below has an ECN of 8.3, and is thus a medium chain oil.

| | | ECN as % of oil | ECN as % of oil |
|---|---|---|---|
| Fatty-acid | % of | (chain length) × | normalized to |
| ChainLength | oil | (% in oil) | monoglyceride |
| C8 monoglyceride | 47 | 8 × 0.47 = 3.76 | 3.76 |
| C10 monoglyceride | 8 | 10 × 0.08 = 0.8 | 0.8 |
| C8 diglyceride | 38 | 2 × (8 × 0.38) = 6.08 | 6.08/2 = 3.04 |
| C10 diglyceride | 7 | 2 × (10 × 0.07) = 1.4 | 1.4/2 = 0.7 |
| OIL ECN (normalized | 8.3 | | |
| to monoglycerides) | | | |

Expressed differently, ECN can be calculated as each chain length in the composition multiplied by its relative percentage in the oil: (8*0.85)+(10*0.15)=8.3.

The term “polysorbate” refers to a compound having the structure:

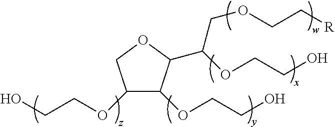

wherein w+x+y+z ranges from about 10 to about 50, and in particular embodiments, from about 10 to about 30, and wherein R is a C6-C18 fatty acid radical. Exemplary polysorbates within the scope of the present definition include, but are not limited to, polysorbate 20, polysorbate 40, polysorbate 60, polysorbate 65, and polysorbate 80.

The term “excipients,” as used herein, refers to non-API ingredients such as solubilizing agents, anti-oxidants, oils, lubricants, dissolution aids, terpenes, and others used in formulating pharmaceutical products.

The phrase “therapeutically effective amount” refers to an amount of a pharmaceutical composition or of a given steroid hormone suitable to treat a particular symptom, disorder, or disease.

As used herein, the phrase “substantially pure” means that an identified component is at least about 90% pure by weight, in certain embodiments, at least about 95% pure by weight, and in still further embodiments, at least about 98% pure by weight.

As used herein, the phrase “steroid hormone” refers to progesterone, 17-hydroxyprogesterone, 5α-dihydroprogesterone, and estradiol.

As used herein, the term “d-limonene” refers to (4R)-1-methyl-4-(1-methylethenyl)-cyclohexene (CAS No. 5989-27-5), which is also known by synonyms including (+)-4-isopropenyl-1-methylcyclohexene, (+)-p-mentha-1,8-diene, and (R)-(+)-Limonene.

As used herein, the phrase “reference product” refers to PROMETRIUM for progesterone and ESTRACE tablets for estradiol, unless otherwise specified.

### Pharmaceutical Compositions

The pharmaceutical compositions disclosed herein are capable of fully solubilizing steroid hormones, and in particular, estradiol and progesterone. Surprisingly, the pharmaceutical compositions in this disclosure provide a significantly better pharmacokinetic (“PK”) profile for steroid hormones, and for estradiol and progesterone in particular, in a subject in need thereof than currently marketed pharmaceutical compositions, such as ESTRACE for estradiol and PROMETRIUM for progesterone. For progesterone, the present pharmaceutical compositions achieve this enhanced PK profile despite containing from about ⅙ to about ⅛ as much progesterone as a comparable volume of PROMETRIUM. PROMETRIUM, for example, contains approximately 400 mg of progesterone per gram of formulation, while the pharmaceutical compositions provided in this disclosure contain, in certain embodiments, from about 10 to about 100 mg progesterone per gram of pharmaceutical composition, and in certain embodiments, about 60 mg progesterone per gram of pharmaceutical composition. Thus, and by way of example only, if a human subject were administered a 500 mg gel cap (a common gelcap size) of PROMETRIUM or a gelcap containing 500 mg of a pharmaceutical compositions disclosed herein comprising about 6 weight percent progesterone, the PROMETRIUM dose would contain 200 mg of progesterone compared to only 30 mg of progesterone in the exemplary pharmaceutical composition. Thus, the human receiving the exemplary composition would receive significantly less progesterone than the subject dosed with PROMETRIUM. Despite the discrepancy in the amount of progesterone dosed, it has now been surprisingly found, that the present compositions provide significantly increased bioavailability compared to PROMETRIUM. The enhanced bioavailability of progesterone and/or estradiol in the present composition allows for a significant reduction in the amount progesterone and/or estradiol that must be administered to a subject per dose to achieve the same or better results as PROMETRIUM and/or ESTRACE.

Without wishing to be bound by any particular theory, it is believed that, in certain embodiments, the described pharmaceutical compositions form micelles upon administration that both protect the estradiol and progesterone from the digestive milieu and facilitate absorption of the estradiol and progesterone across the gut mucosa and into the blood stream. That said, in other embodiments, and without wishing to be bound by any particular theory, the enhanced bioavailability observed in all of the present compositions may be due to the fully-solubilized nature of the estradiol and the progesterone present in the compositions and the absence of suspended (insoluble) estradiol and progesterone, especially progesterone. Thus, in some embodiments, the pharmaceutical composition can be characterized as a fully-solubilized estradiol and fully-solubilized progesterone pharmaceutical composition capable of forming micelles. Other embodiments, however, may comprise fully-solubilized estradiol and fully-solubilized progesterone, but may not form micelles. In still other embodiments, the presence of both fully-solubilized estradiol and fully solubilized progesterone and the formation of micelles together in the same pharmaceutical composition may result in an effect that further enhances the bioavailability of the estradiol and the progesterone above the bioavailability that would result if either only micelles were formed, or only fully-solubilized estradiol and fully-solubilized progesterone were present.

### Compositions Capable of Forming Micelles

In certain embodiments, this disclosure provides a pharmaceutical composition providing enhanced oral bioavailability of a steroid hormone, such as estradiol and/or progesterone, wherein the pharmaceutical composition comprises estradiol, progesterone, a polysorbate, and a medium chain oil.

In certain embodiments, the medium chain oil in the pharmaceutical composition can comprise at least about 50 weight percent of a medium chain monoglyceride. In certain embodiments, the medium chain monoglyceride and the estradiol combined with the progesterone (i.e., estradiol plus progesterone) can be present at a weight ratio of about 8:1 to about 15:1, about 9:1 to about 15:1, about 9:1 to about 14:1, about 9:1 to about 13:1, about 9:1 to about 12:1, about 9:1 to about 11:1, or about 10:1. In particular embodiments, the medium chain monoglyceride and the estradiol combined with the progesterone (i.e., estradiol plus progesterone) can be present in a ratio of about 10:1.

In certain embodiments, the polysorbate can comprise from about 1 weight percent to about 15 weight percent of the pharmaceutical composition and in particular embodiments, can be about 1 weight percent, about 2 weight percent, about 3 weight percent, about 4 weight percent, about 5 weight percent, about 6 weight percent, about 7 weight percent, about 8 weight percent, about 9 weight percent, about 10 weight percent, about 11 weight percent, about 12 weight percent, about 13 weight percent, about 14 weight percent, or about 15 weight percent of the pharmaceutical composition. In particular embodiments, the polysorbate can be about 5 weight percent or about 7 weight percent of the pharmaceutical composition.

In certain embodiments, the steroid hormones, such as estradiol and progesterone, and the polysorbate present in the pharmaceutical composition are present in a weight ratio of about 1:2 to about 2:1.

In certain embodiments, the polysorbate can be selected from the group consisting of polysorbate 20, polysorbate 40, polysorbate 60, polysorbate 65, and polysorbate 80. These polysorbates are commercially available and well known to those of skill in the art. In certain embodiments, the polysorbate can be polysorbate 80. In even more particular embodiments, the polysorbate 80 can comprise about 5 weight percent or about 7 weight percent of the pharmaceutical composition.

In certain embodiments, the medium chain oil can comprise from about 50 weight percent to about 90 weight percent of the pharmaceutical composition. In particular embodiments, the medium chain oil can comprise from about 60 weight percent to about 90 weight percent, from about 65 weight percent to about 90 weight percent, from about 70 weight percent to about 90 weight percent, from about 75 weight percent to about 90 weight percent, or from about 75 weight percent to about 85 weight percent. In particular embodiments, the medium chain oil can comprise about 80 weight percent or about 85 weight percent of the pharmaceutical composition.

In certain embodiments, the medium chain oil can comprise a single medium chain oil component. In other embodiments, the medium chain oil can comprise a first medium chain oil component and a second medium chain oil component. In still further embodiments, the medium chain oil can comprise a first medium chain oil component, a second medium chain oil component, and a third medium chain oil component. In still further embodiments, the medium chain oil can comprise first, second, third and fourth; first, second, third, fourth, and fifth; or first, second, third, fourth, fifth, and sixth medium chain oil components.

In certain embodiments, the medium chain oil components themselves can be multi-component oils. For example, certain medium chain oils comprise a mixture of mono and diglycerides or a mixture of mono-, di-, and triglycerides, etc.

In particular embodiments, the medium chain oil can comprise a first medium chain oil component and a second medium chain oil component, with the first medium chain oil component comprising from about 30 weight percent to about 98 weight percent of the medium chain oil. In other embodiments, the first medium chain oil component can comprise from about 40 weight percent to about 95 weight percent of the medium chain oil. In still further embodiments, the first medium chain oil component can comprise from about 50 weight percent to about 90 weight percent of the medium chain oil.

In certain embodiments, the medium chain oil can comprise at least about 50 weight percent of a medium chain monoglyceride. In particular embodiments, the medium chain oil can comprise at least about at least about 55 weight percent of a medium chain monoglyceride, at least about at least about 60 weight percent of a medium chain monoglyceride, at least about 65 weight percent of a medium chain monoglyceride, at least about 70 weight percent of a medium chain monoglyceride, at least about 71 weight percent of a medium chain monoglyceride, at least about 72 weight percent of a medium chain monoglyceride, at least about 73 weight percent of a medium chain monoglyceride, at least about 74 weight percent of a medium chain monoglyceride, at least about 75 weight percent of a medium chain monoglyceride, at least about 76 weight percent of a medium chain monoglyceride, at least about 77 weight percent of a medium chain monoglyceride, at least about 78 weight percent of a medium chain monoglyceride, at least about 79 weight percent of a medium chain monoglyceride, at least about 80 weight percent of a medium chain monoglyceride, at least about 81 weight percent of a medium chain monoglyceride, at least about 82 weight percent of a medium chain monoglyceride, at least about 83 weight percent of a medium chain monoglyceride, at least about 84 weight percent of a medium chain monoglyceride, at least about 85 weight percent of a medium chain monoglyceride, at least about 86 weight percent of a medium chain monoglyceride, at least about 87 weight percent of a medium chain monoglyceride, at least about 88 weight percent of a medium chain monoglyceride, at least about 89 weight percent of a medium chain monoglyceride, or at least about 90 weight percent of a medium chain monoglyceride. In certain embodiments, the medium chain oil can comprise at least about 85 weight percent of a medium chain monoglyceride and in an even further embodiment, the medium chain oil can comprise at least about 90 weight percent of a medium chain monoglyceride.

The medium chain monoglyceride can be, predominantly, a single medium chain monoglyceride, such as glyceryl monocaproate, glyceryl monocaprylate, glyceryl monocaprate, glyceryl monolaurate, or glyceryl monomyristate. These monoglycerides are well known to those of ordinary skill in the art and are available in various commercial embodiments, including from ABITEC Corp, a division of Associated British Food, PLC, as CAPMUL 708G, CAPMUL 808G, CAPMUL MCM C8, and CAPMUL MCM C10. In particular embodiments, the medium chain monoglyceride can be, predominantly, a single medium chain monoglyceride such as glyceryl monocaproate, glyceryl monocaprylate, or glyceryl monocaprate. In specific embodiments, the medium chain monoglyceride can be, predominantly, glyceryl monocaprylate, commercially available as CAPMUL 708G.

In other embodiments, the medium chain monoglyceride can comprise a mixture of medium chain monoglycerides, such as a combination of two or more of glyceryl monocaproate, glyceryl monocaprylate, glyceryl monocaprate, glyceryl monolaurate, or glyceryl monomyristate. In particular embodiments, the mixture of medium chain monoglycerides can be a mixture of glyceryl monocaprylate and glyceryl monocaprate. In embodiments such as this, the glyceryl monocaprylate can comprise at least about 80 weight percent, at least about 85 weight percent, at least about 86 weight percent, at least about 87 weight percent, at least about 88 weight percent, at least about 88 weight percent, at least about 89 weight percent, or at least about 90 weight percent of the mixture of monoglycerides.

In certain embodiment, in addition to comprising a medium chain monoglyceride, the medium chain oil can further comprise one or more medium chain diglycerides. The one or more medium chain diglycerides can be simple diglycerides, such as glyceryl dicaproate, glyceryl dicaprylate, glyceryl dicaprate, glyceryl dilaurate, or glyceryl dimyristate. Alternatively, the one or more medium chain diglycerides can be mixed or complex diglycerides such as glyceryl caproate/caprylate, glyceryl caproate/caprate, glyceryl caproate/laurate, glyceryl caproate/myristate, glyceryl caprylate/caprate, glyceryl caprylate/laurate, glyceryl caprylate/myristate, glyceryl caprate/laurate, glyceryl caprate/myristate, or glyceryl laurate/myristate. In specific embodiments, the one or more medium chain diglycerides can be glyceryl caprylate/caprate. Exemplary commercially available medium chain diglycerides include, but are not limited to, CAPMUL 471, CAPMUL MCM, CAPMUL MCM NF, CAPMUL MCM EP, and IMWITOR 742. The CAPMULs are commercially available from ABITEC Corp.

The one or more medium chain diglycerides can comprise up to about 10 weight percent of the medium chain oil or alternatively from about 5 to about 10 weight percent of the pharmaceutical composition. In particular embodiments, the one or more medium chain diglycerides can comprise about 5 weight percent, about 6 weight percent, about 7 weight percent, about 8 weight percent, about 9 weight percent, or about 10 weight percent of the pharmaceutical composition. In specific embodiments, the one or more medium chain diglycerides can comprise about from about 8 to about 9 weight percent of the pharmaceutical composition, such as about 8 weight percent, about 8.1 weight percent, about 8.2 weight percent, about 8.3 weight percent, about 8.4 weight percent, about 8.5 weight percent, about 8.6 weight percent, about 8.7 weight percent, about 8.8 weight percent, about 8.9 weight percent, or about 9 weight percent of the pharmaceutical composition.

In certain embodiments, the one or more medium chain diglycerides can be glyceryl caprylate/caprate and the pharmaceutical composition can comprise about 8 weight percent to about 9 weight percent of this component. A commercially available component suitable for use in the pharmaceutical composition is CAPMUL MCM NF.

Because of the manner in which they are prepared, medium chain oils often contain some amount of material that is greater in length than C14. That said, this fraction is typically small and does not affect the overall performance of a given medium chain oil. As such, and in certain embodiments, the amount of material greater than C14 in a given medium chain oil comprises less than 20 weight percent, less than 15 weight percent, less than 5 weight percent, less than 2.5 weight percent, less than 1 weight percent, less than 0.5 weight percent, or less than 0.1 weight percent of a given medium chain oil.

Similarly, monoglycerides and diglycerides often contain di- and triglyceride components in the case of a monoglyceride or mono- and triglycerides in the case of diglycerides. The quantity of these components in a given mono- or diglyceride can vary, but is typically less than 20 weight percent, less than 15 weight percent, less than 5 weight percent, less than 2.5 weight percent, less than 1 weight percent, less than 0.5 weight percent, or less than 0.1 weight percent of the mono- or diglyceride. In all cases, the average ECN will be in the range of 12-28 for diglycerides and 6-14 for monoglycerides.

In addition to the components noted above, in certain embodiments, the pharmaceutical composition can optionally further include a polyoxyethylene hydrogenated castor oil. In particular embodiments, the polyoxyethylene hydrogenated castor oil can be referred to as a “PEG (or polyoxyl) X Hydrogenated Castor Oil,” wherein X refers to the amount of pegylation. In particular embodiments, X can be a number from 1 to 100 and in certain embodiments, can be 7, 40, 40-45, or 60. Exemplary commercially available PEG/polyoxyl X hydrogenated castor oils include CREMOPHOR EL, CREMOPHOR RH40 (available commercially from BASF as polyoxyl 40 hydrogenated castor oil (also known as KOLLIPHOR RH 40)), ETOCAS 40, CRODURET 7, CRODURET 40, CRODURET 50, CRODURET 60, and KOLLIPHOR HS 15. In particular embodiments, the polyoxyethylene hydrogenated castor oil can be polyoxyl 40 hydrogenated castor oil (KOLLIPHOR RH 40).

When present, the polyoxyethylene hydrogenated castor oil can comprise from about 1 to about 10 weight percent of the pharmaceutical composition. In particular embodiments, the polyoxyethylene hydrogenated castor oil can comprise from about 2 to about 9 weight percent of the pharmaceutical composition, from about 3 to about 7 weight percent of the pharmaceutical composition, from about 4 to about 6 weight percent of the pharmaceutical composition, or about 4 to about 5 weight percent of the pharmaceutical composition. In particular embodiments, the polyoxyethylene hydrogenated castor oil can comprise about 4 weight percent of the pharmaceutical composition, about 4.1 weight percent of the pharmaceutical composition, about 4.2 weight percent of the pharmaceutical composition, about 4.3 weight percent of the pharmaceutical composition, about 4.4 weight percent of the pharmaceutical composition, about 4.5 weight percent of the pharmaceutical composition, about 4.6 weight percent of the pharmaceutical composition, about 4.7 weight percent of the pharmaceutical composition, about 4.8 weight percent of the pharmaceutical composition, or about 4.9 weight percent of the pharmaceutical composition.

In other embodiments, the pharmaceutical composition can optionally include a d-α-tocopherol polyethylene glycol succinate (TPGS) derivative having the formula:

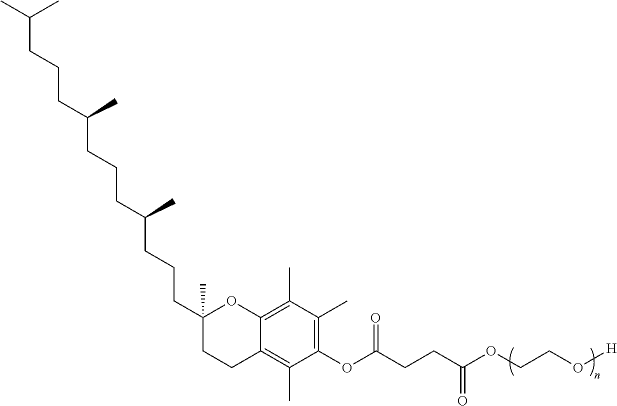

wherein n can range from 1 to about 100, and in particular embodiments, from about 1 to about 50 or about 1 to about 25. In particular embodiments, the d-α-tocopherol polyethylene glycol succinate derivative can be d-α-tocopherol polyethylene glycol 1000 succinate, also referred to as TPGS-1000 (n 22).

The d-α-tocopherol polyethylene glycol succinate derivative, when present, can comprise from about 0.1 weight percent to about 5 weight percent of the pharmaceutical composition and in particular embodiments about 1 weight percent, about 1.5 weight percent, about 1.75 weight percent, about 2 weight percent, about 2.1 weight percent, about 2.2 weight percent, about 2.3 weight percent, about 2.4 weight percent, about 2.5 weight percent, about 2.75 weight percent, about 3 weight percent, about 3.25 weight percent, about 3.5 weight percent, about 3.75 weight percent, about 4 weight percent, about 4.25 weight percent, about 4.5 weight percent, or about 4.75 weight percent of the pharmaceutical composition. In certain embodiments, the d-α-tocopherol polyethylene glycol succinate derivative can comprise about 2.3 weight percent of the pharmaceutical composition. In other embodiments, the pharmaceutical composition can comprise TPGS-1000 at about 2.3 weight percent.

Generally speaking, and in certain embodiments, when the pharmaceutical composition includes a d-α-tocopherol polyethylene glycol succinate derivative, the pharmaceutical composition does not include a polyoxyethylene hydrogenated castor oil. Similarly, and in certain embodiments, when the pharmaceutical composition includes a polyoxyethylene hydrogenated castor oil, the pharmaceutical composition does not include a d-α-tocopherol polyethylene glycol succinate derivative.

As noted at the outset, the pharmaceutical compositions of the present disclosure are believed to form micelles after oral administration. Micelle formation can be observed by adding the pharmaceutical compositions described herein to water or other aqueous-based fluid such as simulated gastric fluid (SGF). The size or size distribution of the micelles resulting from mixing the pharmaceutical compositions with water or SGF can be measured using photon correlation spectroscopy. In certain embodiments, the particles can have a size distribution ranging from about 1 nm to about 1400 nm in water, or from about 130 nm to about 465 nm in water, or from about 100 nm to about 210 nm in water.

In certain embodiments, the micelles can have a zeta potential (mV) ranging from about −10 to about −30 mV. In certain embodiments, the zeta potential of the micelles can be about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, or about −30 mV. In certain embodiments, the zeta potential can be about −16 to about −17 mV. In other embodiments, the zeta potential can be about −18 to about −19 mV. In still other embodiments, the zeta potential can be about −20 to about −21 mV.

### Non-Micelle Forming Pharmaceutical Compositions

In another embodiment, the present disclosure provides non-micelle forming, fully-solubilized steroid hormone pharmaceutical compositions. These compositions comprise estradiol, progesterone and a medium chain oil, wherein the estradiol and the progesterone are both fully solubilized.

In certain embodiments, the medium chain oil in the pharmaceutical composition can comprise at least about 50 weight percent of a medium chain monoglyceride. In certain embodiments, the medium chain monoglyceride, and the estradiol and progesterone (E+P) can be present at a weight ratio of about 8:1 to about 15:1, about 9:1 to about 15:1, about 9:1 to about 15:1, about 10:1 to about 15:1, about 11:1 to about 15:1, about 12:1 to about 15:1, about 13:1 to about 15:1, or about 14:1. In particular embodiments, the medium chain monoglyceride, and the estradiol and progesterone (E+P) can be present in a ratio of about 14:1.

In certain embodiments of the non-micelle forming pharmaceutical composition, the medium chain oil can comprise from about 50 weight percent to about 95 weight percent of the pharmaceutical composition. In particular embodiments, the medium chain oil can comprise from about 60 weight percent to about 95 weight percent, from about 65 weight percent to about 95 weight percent, from about 70 weight percent to about 95 weight percent, from about 75 weight percent to about 95 weight percent, or from about 85 weight percent to about 95 weight percent. In particular embodiments, the medium chain oil can comprise about 94 weight percent of the non-micelle forming pharmaceutical composition.

In certain embodiments, the medium chain oil can comprise a single medium chain oil component. In other embodiments, the medium chain oil can comprise a first medium chain oil component and a second medium chain oil component. In still further embodiments, the medium chain oil can comprise a first medium chain oil component, a second medium chain oil component, and a third medium chain oil component. In still further embodiments, the medium chain oil can comprise first, second, third and fourth; first, second, third, fourth, and fifth; or first, second, third, fourth, fifth, and sixth medium chain oil components.

In certain embodiments, the medium chain oil components themselves can be multi-component oils. For example, certain medium chain oils comprise a mixture of mono and diglycerides or a mixture of mono-, di-, and triglycerides, etc.

In particular embodiments, the medium chain oil can comprise a first medium chain oil component and a second medium chain oil component, with the first medium chain oil component comprising from about 30 weight percent to about 98 weight percent of the medium chain oil. In other embodiments, the first medium chain oil component can comprise from about 40 weight percent to about 95 weight percent of the medium chain oil. In still further embodiments, the first medium chain oil component can comprise from about 50 weight percent to about 90 weight percent of the medium chain oil.

In certain embodiments, the medium chain oil can comprise at least about 50 weight percent of a medium chain monoglyceride. In particular embodiments, the medium chain oil can comprise at least about at least about 55 weight percent of a medium chain monoglyceride, at least about at least about 60 weight percent of a medium chain monoglyceride, at least about 65 weight percent of a medium chain monoglyceride, at least about 70 weight percent of a medium chain monoglyceride, at least about 71 weight percent of a medium chain monoglyceride, at least about 72 weight percent of a medium chain monoglyceride, at least about 73 weight percent of a medium chain monoglyceride, at least about 74 weight percent of a medium chain monoglyceride, at least about 75 weight percent of a medium chain monoglyceride, at least about 76 weight percent of a medium chain monoglyceride, at least about 77 weight percent of a medium chain monoglyceride, at least about 78 weight percent of a medium chain monoglyceride, at least about 79 weight percent of a medium chain monoglyceride, at least about 80 weight percent of a medium chain monoglyceride, at least about 81 weight percent of a medium chain monoglyceride, at least about 82 weight percent of a medium chain monoglyceride, at least about 83 weight percent of a medium chain monoglyceride, at least about 84 weight percent of a medium chain monoglyceride, at least about 85 weight percent of a medium chain monoglyceride, at least about 86 weight percent of a medium chain monoglyceride, at least about 87 weight percent of a medium chain monoglyceride, at least about 88 weight percent of a medium chain monoglyceride, at least about 89 weight percent of a medium chain monoglyceride, at least about 90 weight percent of a medium chain monoglyceride, or at least about 90 weight percent of a medium chain monoglyceride. In certain embodiments, the medium chain oil can comprise at least about 90 weight percent of a medium chain monoglyceride.

The medium chain monoglyceride can be, predominantly, a single medium chain monoglyceride, such as glyceryl monocaproate, glyceryl monocaprylate, glyceryl monocaprate, glyceryl monolaurate, or glyceryl monomyristate. These monoglycerides are well known to those of ordinary skill in the art and are available in various commercial embodiments, including from ABITEC Corp, a division of Associated British Food, PLC, as CAPMUL 708G, CAPMUL 808G, CAPMUL MCM C8, and CAPMUL MCM C10. In particular embodiments, the medium chain monoglyceride can be, predominantly, a single medium chain monoglyceride such as glyceryl monocaproate, glyceryl monocaprylate, or glyceryl monocaprate. In specific embodiments, the medium chain monoglyceride can be, predominantly, glyceryl monocaprylate, commercially available as CAPMUL 708G.

In other embodiments, the medium chain monoglyceride can comprise a mixture of medium chain monoglycerides, such as a combination of two or more of glyceryl monocaproate, glyceryl monocaprylate, glyceryl monocaprate, glyceryl monolaurate, or glyceryl monomyristate. In particular embodiments, the mixture of medium chain monoglycerides can be a mixture of glyceryl monocaprylate and glyceryl monocaprate. In embodiments such as this, the glyceryl monocaprylate can comprise at least about 80 weight percent, at least about 85 weight percent, at least about 86 weight percent, at least about 87 weight percent, at least about 88 weight percent, at least about 88 weight percent, at least about 89 weight percent, or at least about 90 weight percent of the mixture of monoglycerides.

In certain embodiment, in addition to comprising a medium chain monoglyceride, the medium chain oil can further comprise one or more medium chain diglycerides. The one or more medium chain diglycerides can be simple diglycerides, such as glyceryl dicaproate, glyceryl dicaprylate, glyceryl dicaprate, glyceryl dilaurate, or glyceryl dimyristate. Alternatively, the one or more medium chain diglycerides can be mixed or complex diglycerides such as glyceryl caproate/caprylate, glyceryl caproate/caprate, glyceryl caproate/laurate, glyceryl caproate/myristate, glyceryl caprylate/caprate, glyceryl caprylate/laurate, glyceryl caprylate/myristate, glyceryl caprate/laurate, glyceryl caprate/myristate, or glyceryl laurate/myristate. In specific embodiments, the one or more medium chain diglycerides can be glyceryl caprylate/caprate. Exemplary commercially available medium chain diglycerides include, but are not limited to, CAPMUL 471, CAPMUL MCM, CAPMUL MCM NF, CAPMUL MCM EP, and IMWITOR 742. The CAPMULs are commercially available from ABITEC Corp.

The one or more medium chain diglycerides can comprise up to about 10 weight percent of the medium chain oil or alternatively from about 5 to about 10 weight percent of the pharmaceutical composition. In particular embodiments, the one or more medium chain diglycerides can comprise about 5 weight percent, about 6 weight percent, about 7 weight percent, about 8 weight percent, about 9 weight percent, or about 10 weight percent of the pharmaceutical composition. In specific embodiments, the one or more medium chain diglycerides can comprise about from about 8 to about 9 weight percent of the pharmaceutical composition, such as about 8 weight percent, about 8.1 weight percent, about 8.2 weight percent, about 8.3 weight percent, about 8.4 weight percent, about 8.5 weight percent, about 8.6 weight percent, about 8.7 weight percent, about 8.8 weight percent, about 8.9 weight percent, or about 9 weight percent of the pharmaceutical composition.

In certain embodiments, the one or more medium chain diglycerides can be glyceryl caprylate/caprate and the pharmaceutical composition can comprise about 8 weight percent to about 10 weight percent of this component. A commercially available component suitable for use in the pharmaceutical composition is CAPMUL MCM NF.

### Excluded Ingredients

In preferred embodiments, the various pharmaceutical compositions described in this disclosure are completely or substantially free of animal oils, vegetable oils, and fractionated vegetable oils. Exemplary excluded animal oils include, but are not limited to, fish liver oils, shark oil, and mink oil. Exemplary excluded fractionated vegetable oils include, but are not limited to, fractionated coconut oils. Exemplary excluded vegetable oils include soy bean oil, peanut oil, safflower seed oil, corn oil, olive oil, cottonseed oil, arachis oil, sunflower seed oil, coconut oil, palm oil, and rape seed oil. In embodiments, the pharmaceutical compositions described in this disclosure are completely or substantially free of all omega-3 free fatty acids and all omega-3 fatty acid esters, including, for example, hexadecatrienoic acid, .alpha.-linolenic acid, stearidonic acid, eicosatrienoic acid, eicosapentaenoic acid, heneicosapentaenoic acid, docosapentenoic acid, docosahexaenoic acid, tetracosapentenoic acid, tetracosahexaenoic acid, or combinations thereof. In preferred embodiments, the pharmaceutical compositions described herein are completely or substantially free of EPA fatty acid esters and DHA fatty acid esters.

### Steroid Hormone

In certain embodiments, the pharmaceutical composition can comprise from about 0.025 weight percent to about 15 weight percent of a steroid hormone or a combination of steroid hormones. In certain embodiments, the pharmaceutical composition can comprise from about 0.025 weight percent to about 10 weight percent of estradiol and progesterone (e.g., estradiol plus progesterone), from about 1 to about 10 weight percent estradiol and progesterone, about 1 to about 9 weight percent estradiol and progesterone, from about 1 to about 8 weight percent estradiol and progesterone, from about 1 to about 7 weight percent estradiol and progesterone, from about 2 to about 7 weight percent estradiol and progesterone, from about 3 to about 7 weight percent estradiol and progesterone, from about 4 to about 7 weight percent estradiol and progesterone, from about 5 to about 7 weight percent estradiol and progesterone, or about 6 weight percent estradiol and progesterone.

The steroid hormone, and in particular embodiments, estradiol and/or progesterone, can be partially solubilized (i.e. less than about 80% solubilized), solubilized, or fully solubilized, depending upon the specific components of the composition. In typical embodiments, the steroid hormones, i.e., estradiol and progesterone, are at least solubilized and in certain embodiments, fully, i.e., completely, solubilized in the pharmaceutical composition. In some embodiments, the pharmaceutical composition is saturated such that additional steroid hormone, i.e., estradiol and/or progesterone, will not dissolve. In some embodiments, the pharmaceutical composition contains both solubilized and suspended (insoluble) estradiol and/or progesterone. That said, and more typically, the estradiol and progesterone are each at least about 80%, at least about 85%, at least about 90%, at least about 95%, at least about 99%, or 100% solubilized in the pharmaceutical composition at a given concentration. In certain embodiments, the estradiol and the progesterone are each fully solubilized, i.e., at least about 95 percent solubilized, at least about 98% solubilized, or at least about 99% solubilized as measured according to the methodology described elsewhere herein. However, in other embodiments, the progesterone can be solubilized or only partially solubilized.

The solubility of a given steroid hormone can be measured using standard techniques by weighing a piece of filter paper, placing the weighed filter paper in a buchner funnel (porcelain or glass with a glass frit), and drawing a known quantity of pharmaceutical composition through the filter paper using vacuum (such as with a side-arm flask fitted with a neoprene collar). After drying for an appropriate period of time (either at room temperature or at elevated temperature), the filter paper is reweighed. The amount of steroid hormone on the filter paper is calculated and the amount or percentage of solubilized and insoluble steroid hormone is calculated.

In certain embodiments, the bio-identical steroid hormone estradiol can be replaced with estrone, estriol or estrogen analogs. Similarly, the bio-identical steroid hormone progesterone can be replaced with progesterone analogs. In preferred embodiments, one (and ideally both) of the steroid hormones is a bio-identical steroid hormone.

Although the estradiol and progesterone used to formulate the pharmaceutical compositions can have any particle size, in certain embodiments, the estradiol and progesterone can each have an average particle size of less than about 100 microns. In certain embodiments, one or both of the estradiol and the progesterone can be micronized. Without wishing to be bound by any particular theory, it is believed that steroid hormones having a smaller average particle size will be more soluble in the pharmaceutical compositions.

### Terpenes

All of the pharmaceutical compositions described in this disclosure can optionally include a terpene. Terpenes are the primary constituents of the essential oils of many types of plants and flowers and are typically formed directly from one or more isoprene (C₅H₈) units. Terpenes can be naturally occurring or prepared synthetically. Terpenes can be obtained from their natural source, for example, isolated from a natural oil such as citrus oil or orange oil, and optionally purified to be substantially pure, or synthesized chemically.

In certain embodiments, the terpene can be a terpenoid. Examples of terpenes are provided, for example, in Dev et al., “CRC Handbook of Terpenoids: Acyclic, Monocyclic, Bicyclic, Tricyclic, and Tetracyclic Terpenoids” (1989) CRC Press Inc.; Hanson, J. R., Annu. Rep. Prog. Chem., Sect. B: Org. Chem., (1985) 82, 353-375; and Degenhardt et al., Phytochemistry (2009) 70:1621-1637. Each of these references is hereby incorporated by reference in its entirety.

The optional terpene can be linear or cyclic (including aromatic). A cyclic terpene can be a monocyclic terpene or a bicyclic terpene. In a particular embodiment, the cyclic terpene can be a monocyclic terpene. In certain embodiments, the cyclic terpene can be non-aromatic. Examples of cyclic terpenes include, without limitation, limonene (as d-limonene, l-limonene, or a mixture thereof), phellandrene (alpha or beta), camphor, menthol, menthene, carvone, terpinene (alpha, beta, or gamma), terpineol (alpha, beta, or gamma), alpha-ionone, thujone, and derivatives thereof. In certain embodiments, the cyclic terpene is limonene, menthene, menthol, phellandrene, terpinene, or terpineol. In some embodiments, the optional terpene can be d-limonene.

In certain embodiments, when the terpene is present, the terpene can comprise from about 0.5 weight percent to about 10 weight percent of the pharmaceutical composition; from about 1 weight percent to about 10 weight percent of the pharmaceutical composition; from about 2 weight percent to about 9 weight percent of the pharmaceutical composition; from about 3 weight percent to about 8 weight percent of the pharmaceutical composition; from about 4 weight percent to about 8 weight percent of the pharmaceutical composition; from about 5 weight percent to about 7 weight percent of the pharmaceutical composition, or about 6 weight percent of the pharmaceutical composition.

In certain embodiments, the optional terpene is d-limonene and is present in any of the amounts noted above. In other embodiments, the optional terpene is d-limonene and is present at about 6 weight percent of the pharmaceutical composition.

### Antioxidants

In certain embodiments, the pharmaceutical compositions can further include an antioxidant such as α-tocopherol acetate, acetone sodium bisulfite, acetylcysteine, ascorbic acid, ascorbyl palmitate, butylated hydroxyanisole (BHA), butylated hydroxytoluene (BHT), cysteine, cysteine hydrochloride, α-tocopherol, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, tocopherol, or any combination thereof. In particular embodiments, the antioxidant is BHT.

The antioxidant can be included in an amount appropriate to inhibit oxidation of any, some, or all of the components of the pharmaceutical composition for a desired period of time. For example, the antioxidant can inhibit oxidation of any of the estradiol and/or progesterone present in the pharmaceutical composition, the medium chain oil, the polysorbate, the polyoxyethylene hydrogenated castor oil, the D-α-Tocopherol polyethylene glycol succinate derivative, or the terpene to the extent these components are present in the composition. In certain embodiments, the antioxidant is present to inhibit the oxidation of the terpene, which in certain embodiments, can be d-limonene. In certain embodiments, the BHT is present in the pharmaceutical composition at from about 0.01 to about 0.1 weight percent. In other embodiments, the BHT is present at about 0.03 weight percent.

### Methods of Treating Hormone Deficiencies

In certain embodiments, this disclosure provides methods for treating one or more conditions associated with hormone deficiency in a subject. The methods comprise orally administering to a subject in need thereof an effective amount of the pharmaceutical composition described herein.

In some embodiments, the condition being treated can be an estrogen deficiency. In some embodiments, the conditions to be treated can be a condition associate with or related to menopause, including hot flashes/flushes, night sweats, sleep disturbances, mood changes, vulvovaginal atrophy, or osteoporosis. In certain embodiments, the condition is a vasomotor symptom of menopause including hot flashes, hot flushes, night sweats, sleep disturbances or mood changes.

Importantly, the progesterone is present in the composition disclosed herein to counteract side effects of estradiol in subjects receiving estradiol therapy.

In some embodiments, the condition being treated can be an estrogen deficiency. In some embodiments, the condition can be hot flashes, night sweats, sleep disturbances, mood changes, vulvovaginal atrophy, or osteoporosis.

In some embodiments, the condition being treated can be a progesterone deficiency. In some embodiments, the condition can be endometrial hyperplasia, secondary amenorrhea, prevention of preterm birth; and osteoporosis. In some embodiments, progesterone is delivered, together with estradiol, to treat vasomotor symptoms of menopause, including, hot flashes, night sweats, sleep disturbances, mood changes, or osteoporosis. In some embodiments, the progesterone deficiency is menopause.

In certain embodiments, the steroid hormone(s) can be present in the pharmaceutical composition or can be administered in the range of about 0.1 mg to about 1 g per dosage unit; about 1 mg to about 600 mg per dosage unit; or about 10 mg to about 500 mg per dosage unit.

In certain embodiments, the estradiol can be present in the pharmaceutical composition or can be administered in the range of about 0.01 mg to about 2 mg, and in certain embodiments, about 2 mg, about 1.5 mg, about 1 mg, about 0.75 mg, about 0.5 mg, about 0.25 mg, about 0.2 mg, about 0.15 mg about 0.1 mg, about 0.075 mg, about 0.050 mg, about 0.025 mg, about 0.01 mg, or any range encompassing any of the noted values.

In certain embodiments, the progesterone can be present in the pharmaceutical composition or can be administered in the range of about 15 mg to about 500 mg, and in certain embodiments, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, or any range encompassing any of the noted values.

In particular embodiments, the amount of progesterone administered per dose using the pharmaceutical composition in this disclosure to a human in need thereof, can range from about 10 mg to about 50 mg or from about 15 mg to about 45 mg. In certain embodiments, the amount of progesterone administered to a subject in need thereof using the pharmaceutical composition of this disclosure can be about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37, about 38 mg, about 39 mg, or about 40 mg progesterone. In particular embodiments, a human in need thereof can receive either about 20 mg progesterone or about 36 mg progesterone when the pharmaceutical composition is administered.

In order to receive the desired amount of estradiol and progesterone per dose, the human in need thereof can, in certain embodiments, be administered from about 300 mg to about 2000 mg of the pharmaceutical composition, from about 350 mg to about 1700 mg of the pharmaceutical composition, from about 400 mg to about 1400 mg of the pharmaceutical composition, from about 450 mg to about 1100 mg of the pharmaceutical composition, from about 500 mg to about 800 mg of the pharmaceutical composition, from about 550 mg to about 750 mg of the pharmaceutical composition, from about 575 mg to about 625 mg of the pharmaceutical composition, or about 600 mg of the pharmaceutical formulation. In other embodiments, the human in need thereof can be administered about 300 to about 350 mg of the pharmaceutical composition. In other embodiments, the human in need thereof can be administered about 350 to about 400 mg of the pharmaceutical composition. In other embodiments, the human in need thereof can be administered about 400 to about 450 mg of the pharmaceutical composition. In other embodiments, the human in need thereof can be administered about 450 to about 500 mg of the pharmaceutical composition. In other embodiments, the human in need thereof can be administered about 500 to about 550 mg of the pharmaceutical composition. In other embodiments, the human in need thereof can be administered about 550 to about 600 mg of the pharmaceutical composition. In other embodiments, the human in need thereof can be administered about 600 to about 650 mg of the pharmaceutical composition.

In embodiments wherein the amount of estradiol and progesterone in the composition is about 6 weight percent of the composition and wherein the amounts of estradiol and progesterone to be administered to the human in need thereof are about 0.01 mg to about 2.0 mg estradiol and about 20 mg to about 40 mg progesterone, the amount of the pharmaceutical formulation that can be administered to the human can be about 300 mg to about 600 mg.

In embodiments wherein the amount of estradiol and progesterone in the composition is about 6 weight percent of the composition and wherein the amount of estradiol and progesterone to be administered to the human in need thereof is about 0.01 to about 2.0 mg estradiol and about 30 to about 42 mg progesterone, the amount of the pharmaceutical formulation that can be administered to the human can be about 450 mg to about 600 mg.

These dosages reflect the surprisingly enhanced bioavailability of estradiol and, in particular, progesterone provided by the present pharmaceutical compositions. These compositions provide the opportunity to reduce the amount of estradiol and/or progesterone administered to a human in need thereof relative to currently marketed products such as ESTRACE and/or PROMETRIUM. As discussed elsewhere herein, the PK parameters observed for progesterone when the present pharmaceutical compositions are dosed are highly surprising in view of the known PK parameters associated with PROMETRIUM.

In certain embodiments, the pharmaceutical compositions comprising both estradiol and progesterone can be administered to a human in need thereof in the amounts described above for the treatment of a disease or conditions treatable with estrogen and, in particular, estradiol. Such diseases and conditions include those related to low estrogen levels and, in particular, menopause. Conditions associated with low estrogen levels include hot flashes, hot flushes, night sweats, sleep disturbances, mood changes, vulvovaginal atrophy, or osteoporosis. Conditions associated with menopause include vasomotor symptoms, such as hot flashes/flushes, night sweats and sleep disturbances. Importantly, the progesterone is present in the composition disclosed herein to counteract side effects of estradiol in subjects receiving estradiol therapy.

In certain embodiments, a human can be administered from about 300 mg to about 650 mg of a pharmaceutical compositions described herein to treat a disease or condition associated with low estrogen levels.

In other embodiments, a human can be administered from about 300 mg to about 650 mg of a pharmaceutical compositions described herein to a vasomotor symptom of menopause, such as hot flashes, night sweats and sleep disturbances.

In other embodiments, a human can be administered from about 300 mg to about 650 mg of a pharmaceutical compositions described herein to treat vulvovaginal atrophy.

In other embodiments, a human can be administered from about 300 mg to about 650 mg of a pharmaceutical compositions described herein to treat osteoporosis.

In each of the above described embodiments, a human can be administered about a dose of about 333 mg or about 600 mg of the pharmaceutical composition, such that the human receives about 20 mg or about 36 mg of progesterone per dose of the pharmaceutical composition.

In certain embodiments, the pharmaceutical composition can be administered once daily within in any of the above noted amounts until the disease or condition is treated.

In further embodiments, about 300 mg to about 600 mg of the pharmaceutical composition can be administered once daily to treat the disease or condition associated with low estrogen levels.

In still another embodiment, about 300 mg to about 600 mg of the pharmaceutical composition can be administered once daily to treat the disease or condition associated with menopause, such as a vasomotor symptom of menopause.

In certain embodiments, the amount of pharmaceutical composition administered to a given human subject can be an amount that renders the pharmaceutical composition bioequivalent to ESTRACE and/or PROMETRIUM.

In certain embodiments, the amount of the pharmaceutical composition that is bioequivalent to PROMETRIUM can be from about 300 to about 350 mg of the pharmaceutical composition. In certain embodiments, the pharmaceutical composition can comprise about 6 weight percent progesterone. In still further embodiments, the amount of progesterone administered to the human subject using the present pharmaceutical compositions to achieve bioequivalence to PROMETRIUM can be about 20 mg progesterone.

Although the pharmacokinetic profiles of many progesterone formulations can be affected by whether or not the formulation is taken with food, it has been surprisingly discovered that, in some embodiments, the present pharmaceutical compositions can deliver progesterone consistently both in the presence and absence of food. That is, and surprisingly, in some embodiments, the present pharmaceutical compositions do not show a food effect. This is an extremely beneficial property of certain embodiments of the disclosed pharmaceutical compositions as it allows for less restrictive dosing and increases the likelihood of patient compliance with a given dosing regimen. Lack of a food effect may further reduce both inter- and intra-patient variability when the pharmaceutical compositions of the present disclosure are dosed.

In certain embodiments, the pharmaceutical composition can be administered once daily until the condition is treated.

### Pharmacokinetics and Metabolites

The disclosed pharmaceutical compositions can provide enhanced pharmacokinetics versus the currently marketed drugs ESTRACE and PROMETRIUM. For example, in certain embodiments, the pharmaceutical composition can have an AUC₀₋ₜ that is at least about 1.1, at least about 1.2, at least about 1.3, at least about 1.4, at least about 1.5, at least about 1.6, at least about 1.7, at least about 1.8, at least about 1.9, or at least about 2 times greater than ESTRACE and/or PROMETRIUM when the pharmaceutical compositions are dosed in the fasting state. That is, the pharmaceutical compositions can have AUCs that are at least about 110 percent, at least about 120 percent, at least about 130 percent, at least about 140 percent, at least about 150 percent, at least about 160 percent, at least about 170 percent, at least about 180 percent, at least about 190 percent, or at least about 200 percent the AUC of ESTRACE and/or PROMETRIUM when the pharmaceutical compositions are dosed in the fasting state.

Similarly, in certain embodiments, the pharmaceutical composition can have an Cₘₐₓ that is at least about 1.1, at least about 1.2, at least about 1.3, at least about 1.4, at least about 1.5, at least about 1.6, at least about 1.7, at least about 1.8, at least about 1.9, at least about 2, at least about 2.2, at least about 2.4, at least about 2.6, at least about 2.8, or at least about 3 times greater than ESTRACE and/or PROMETRIUM when the pharmaceutical compositions are dosed in the fasting state.

In certain embodiments, the pharmaceutical composition can have an tₘₐₓ that is at least about 3, at least about 4, at least about 5, at least about 6, at least about 7, at least about 8, at least about 9, at least about 10, at least about 11, at least about 12, at least about 13, at least about 14, at least about 15, at least about 16, or at least about 17 times shorter than ESTRACE and/or PROMETRIUM when the pharmaceutical compositions are dosed in the fasting state. That is, and in certain embodiments, the pharmaceutical composition disclosed herein reaches its Cₘₐₓ considerably earlier than ESTRACE and/or PROMETRIUM.

### Methods for Preparing the Pharmaceutical Compositions

In certain embodiments, the compositions described herein can be prepared according to the following general procedure. In certain embodiments, and in a first step, the steroid hormone, and in particular embodiments, progesterone, can be solubilized in the medium chain oil by mixing the steroid hormone with the medium chain oil under mild heating, i.e., from about 35° C. to about 60° C., and in certain embodiments at about 40° C. The mixture can be mixed for an amount of time sufficient to solubilize and uniformly distribute the steroid hormone in the medium chain oil. Typically, the solubilization can be performed in an appropriate vessel, such as optionally temperature-controlled jacketed stainless steel vessel of the type typically found in medium and large scale formulation manufacturing facilities.

The medium chain oil can have properties described elsewhere herein and can be added in the amounts specified elsewhere herein. In particular embodiments, the medium chain oil can comprise a mixture of a medium chain monoglyceride and a medium chain diglyceride. In other embodiments, however, the medium chain oil can comprise a medium chain monoglyceride or a medium chain diglyceride.

As discussed elsewhere herein, the medium chain monoglyceride can be, predominantly, a single medium chain monoglyceride, such as glyceryl monocaproate, glyceryl monocaprylate, glyceryl monocaprate, glyceryl monolaurate, or glyceryl monomyristate. These monoglycerides are well known to those of ordinary skill in the art and are available in various commercial embodiments, including from ABITEC Corp, a division of Associated British Food, PLC, as CAPMUL 708G, CAPMUL 808G, CAPMUL MCM C8, and CAPMUL MCM C10. In particular embodiments, the medium chain monoglyceride can be, predominantly, a single medium chain monoglyceride such as glyceryl monocaproate, glyceryl monocaprylate, or glyceryl monocaprate. In specific embodiments, the medium chain monoglyceride can be, predominantly, glyceryl monocaprylate, commercially available as CAPMUL 708G.

Similarly, and also discussed previously, the one or more medium chain diglycerides can be simple diglycerides, such as glyceryl dicaproate, glyceryl dicaprylate, glyceryl dicaprate, glyceryl dilaurate, or glyceryl dimyristate. Alternatively, the one or more medium chain diglycerides can be mixed or complex diglycerides such as glyceryl caproate/caprylate, glyceryl caproate/caprate, glyceryl caproate/laurate, glyceryl caproate/myristate, glyceryl caprylate/caprate, glyceryl caprylate/laurate, glyceryl caprylate/myristate, glyceryl caprate/laurate, glyceryl caprate/myristate, or glyceryl laurate/myristate. In specific embodiments, the one or more medium chain diglycerides can be glyceryl caprylate/caprate. Exemplary commercially available medium chain diglycerides include, but are not limited to, CAPMUL 471, CAPMUL MCM, CAPMUL MCM NF, CAPMUL MCM EP, and IMWITOR 742. The CAPMULs are commercially available from ABITEC Corp.

In certain embodiments, the one or more medium chain diglycerides can be glyceryl caprylate/caprate. A commercially available version of this component suitable for use in the pharmaceutical composition is CAPMUL MCM NF.

Once the steroid hormone has sufficiently dissolved in the medium chain oil, additional components which can be included in a given composition as specified elsewhere herein, can be added. For example, in certain embodiments, a polysorbate can be added to the medium chain oil/steroid hormone mixture in the amounts specified elsewhere herein. In certain embodiments, the polysorbate can be selected from the group consisting of polysorbate 20, polysorbate 40, polysorbate 60, polysorbate 65, and polysorbate 80. In certain embodiments, the polysorbate can be polysorbate 80.

In certain embodiments, and in addition to the polysorbate, a d-α-tocopherol polyethylene glycol succinate (TPGS) derivative and an antioxidant can be added to the medium chain oil/steroid hormone mixture. Each of the d-α-tocopherol polyethylene glycol succinate (TPGS) derivative and an antioxidant can be added in the amounts and specific embodiments discussed elsewhere herein

In other embodiments, in addition to the polysorbate, a PEG X hydrogenated castor oil and an antioxidant can also be added to the medium chain oil/steroid hormone mixture. Each of the PEG X hydrogenated castor oil and an antioxidant can be added in the amounts and specific embodiments discussed elsewhere herein.

In each of the above noted embodiments, addition of the antioxidant can be omitted.

Typically, and when added to a given composition, the various additional components are added with mixing and under mild heating to ensure homogenous distribution of the various components in the composition. When present, each of the antioxidant, the polysorbate, the d-α-tocopherol polyethylene glycol succinate (TPGS) derivative, and the PEG X hydrogenated castor oil, can be added in the amounts and embodiments disclosed elsewhere herein.

Once the addition of all of the necessary or desired components is complete, the composition can be stirred until it reaches room temperature. Once at room temperature, and when desired, a terpene, such as d-limonene, can be added to the composition in any of the amounts specified elsewhere herein.

The resulting composition, after an optional deaeration process, can then be used as the fill material in the encapsulation process disclosed elsewhere herein.

In another embodiment, the compositions described herein may be prepared by mixing the desired components, exclusive of the optional terpene, at room temperature and subsequently warming the resulting mixture to from about 35° C. to about 60° C., and in certain embodiments to about 40° C. to affect dissolution of the steroid hormone. Following a sufficient amount of stirring to ensure the desired level of dissolution and homogenous distribution of the various components in the composition, the mixture can be cooled to room temperature. After cooling, and as in the alternative embodiment discussed above, a terpene, such as d-limonene, can be added to the composition in any of the amounts specified elsewhere herein.

The resulting composition, after an optional deaeration process, can then be used as the fill material in the encapsulation process disclosed elsewhere herein.

### Encapsulation

Although the pharmaceutical composition can be dosed as a liquid, in certain embodiments, the pharmaceutical composition can be encapsulated in a gelatin capsule, or other similar encapsulated dosage form known to those of skill in the art. The gelatin capsule can be a soft gelatin capsule or a hard gelatin capsule. The hard gelatin capsule can be a two-piece, standard gelatin capsule which typically includes a first capsule portion (i.e., half or bottom) and a second capsule portion (i.e., the other half or top). The soft gelatin capsule can be a two-piece capsule wherein two portions are sealed together or a one-piece, hermetically sealed capsule.

In certain embodiments, the soft gelatin capsule can be a one-piece, hermetically sealed gelatin based capsule which can be made by techniques known to those skilled in the art. In certain embodiments, the gelatin used to form the soft gelatin capsule can include water, gelatin, and a plasticizer to control the softness and flexibility of the capsule. Other additives for use in the gelatin suitable for preparing the soft gelatin capsule, include but are not limited to, flavorants, colorants, and opacifiers.

Soft gelatin capsules can be produced in a known manner, including with a rotary die process in which a molten mass of a gelatin containing the appropriate or necessary additives, is fed from a reservoir onto drums to form two spaced sheets or ribbons of gelatin in a semi-molten state. These ribbons are fed around rollers and brought together at convergent angle into the nip of a pair of roller dies that include opposed die cavities. A liquid fill formulation, such as the pharmaceutical composition of this disclosure, can then be fed into the wedge-shaped joinder of the ribbons. The gelatin ribbons are continuously conveyed between the dies, with portions of the fill formulation being trapped between the sheets inside the die cavities. The sheets are then pressed together, and severed around each die so that opposed edges of the sheet flow together to form a continuous gelatin sheath around the entrapped liquid pharmaceutical composition. The part of the gelatin sheet that is severed from the segments forming the capsules can then be collected for recycling or can be discarded. The resulting soft capsules can then be dried and packaged.

Various gelatin compositions known in the prior art can be used to encapsulate the pharmaceutical composition of this disclosure. For example, suitable gelatin capsules can be prepared from a gelatin mixture comprising from about 30% w/w to about 85% w/w gelatin and in certain embodiments, about 30% w/w to about 50% w/w; about 15% w/w to about 40% w/w of one or more plasticizer; and from 25% w/w to about 50% w/w of water. In certain embodiments, the gelatin will have a bloom in the rage of about 150 to about 275, and can be Type A or B gelatins or a mixture thereof.

Examples of suitable Type A gelatin include without limitation acid bone gelatin. Examples of suitable Type B gelatin include without limitation lime bone gelatin.

Suitable gelatin plasticizers are well known to those of ordinary skill in the art and include, but are not limited to, polyhydric alcohols such as sorbitol, glycerin, mannitol, xylitol, maltitol, and sorbitan; dialkylphthalates; lower alkyl citrates wherein the lower alkyl has 1-6 carbon atoms; glycols and polyglycols including polyethylene glycols with a molecular weight range of about 200 to about 2,000, methoxyl-propylene-glycol, and 1,2-propylene glycol; esters of polyhydroxy-alcohols such as mono-, di-, and tri-acetates of glycerol; ricinoleic acid and esters thereof; and mixtures of the above. The gelatin pharmaceutical composition can also contain other ingredients including, but not limited to, taste modifiers, coloring agents, opacifiers, and moisture retaining agents.

### Additional Non-Limiting Embodiments

Additional non-limiting embodiments of the pharmaceutical compositions and methods disclosed herein are described below:
(A) A pharmaceutical composition for providing enhanced oral bioavailability of estradiol and progesterone, the pharmaceutical composition comprising: estradiol, progesterone, a polysorbate, and a medium chain oil, wherein the medium chain oil comprises at least about 50 weight percent of a predominantly medium chain monoglyceride; and the progesterone is fully solubilized.
(B) The pharmaceutical composition of embodiment (A), wherein the medium chain oil comprises at least about 60 weight percent of a predominantly medium chain monoglyceride.
(C) The pharmaceutical composition of embodiment (A), wherein the medium chain oil comprises at least about 70 weight percent of the predominantly medium chain monoglyceride.
(D) The pharmaceutical composition of embodiment (A), wherein the medium chain oil comprises at least about 80 weight percent of the predominantly medium chain monoglyceride.
(E) The pharmaceutical composition of embodiment (A), wherein the medium chain oil comprises at least about 90 weight percent of the predominantly medium chain monoglyceride.
(F) The pharmaceutical composition of embodiment (E), wherein the polysorbate is selected from the group consisting of polysorbate 20, polysorbate 40, polysorbate 60, polysorbate 65, and polysorbate 80.
(G) The pharmaceutical composition of embodiment (A), wherein the pharmaceutical composition provides an oral bioavailability of at least about 150 percent as measured by an increase in AUC when compared to a reference product.
(H) The pharmaceutical composition of embodiment (A), further comprising a polyoxyethylene hydrogenated castor oil.
(I) The pharmaceutical composition of embodiment (A), further comprising a d-α-tocopherol polyethylene glycol succinate derivative.
(J) The pharmaceutical composition of embodiment (A), further comprising a terpene.
(K) The pharmaceutical composition of embodiment (A), wherein the predominantly medium chain monoglyceride and the progesterone can be present at a weight ratio of about 10:1 to about 15:1.
(L) The pharmaceutical composition of embodiment (K), wherein the predominantly medium chain monoglyceride comprises at least about 60 weight percent of the medium chain oil.
(M) The pharmaceutical composition of embodiment (K), wherein the predominantly medium chain monoglyceride comprises at least about 70 weight percent of the predominantly medium chain oil.
(N) The pharmaceutical composition of embodiment (K), wherein the predominantly medium chain monoglyceride comprises at least about 80 weight percent of the medium chain oil.
(O) The pharmaceutical composition of embodiment (A), wherein the predominantly medium chain monoglyceride comprises at least about 90 weight percent of the medium chain oil.
(P) The pharmaceutical composition of embodiment (O), wherein the polysorbate is selected from the group consisting of polysorbate 20, polysorbate 40, polysorbate 60, polysorbate 65, and polysorbate 80.
(Q) The pharmaceutical composition of embodiment (K), wherein the pharmaceutical composition provides an oral bioavailability of at least about 150 percent for estradiol and/or progesterone as measured by an increase in AUC when compared to a reference product, such as ESTRACE or PROMETRIUM.
(R) The pharmaceutical composition of embodiment (K), further comprising a polyoxyethylene hydrogenated castor oil.
(S) The pharmaceutical composition of embodiment (K), further comprising a d-α-tocopherol polyethylene glycol succinate derivative.
(T) The pharmaceutical composition of embodiment (A), further comprising a terpene.
(U) An oral, fully-solubilized estradiol and progesterone pharmaceutical composition comprising: estradiol, progesterone and a polysorbate, wherein the weight ratio of estradiol and progesterone to polysorbate (E+P:polysorbate) is from about 1:2 to about 2:1; and a medium chain oil comprising a mixture of medium chain mono- and diglycerides, the medium chain oil not containing more than about 10 weight percent triglycerides.
(V) The pharmaceutical composition of embodiment (U), wherein the polysorbate is selected from the group consisting of polysorbate 20, polysorbate 40, polysorbate 60, polysorbate 65, and polysorbate 80.
(W) The pharmaceutical composition of embodiment (U), wherein the pharmaceutical composition provides an oral bioavailability for estradiol and/or progesterone of at least about 150 percent as measured by an increase in AUC when compared to a reference product, such as ESTRACE or PROMETRIUM.
(X) The pharmaceutical composition of embodiment (U), further comprising a polyoxyethylene hydrogenated castor oil.
(Y) The pharmaceutical composition of embodiment (U), further comprising a d-α-tocopherol polyethylene glycol succinate derivative.
(Z) The pharmaceutical composition of embodiment (U), further comprising a terpene.
(AA) The pharmaceutical composition of any of embodiments (A) through (T), wherein the predominantly medium chain monoglyceride is glyceryl monocaproate, glyceryl monocaprylate, glyceryl monocaprate, glyceryl monolaurate, or glyceryl monomyristate.
(BB) The pharmaceutical composition of embodiment (AA), wherein the medium chain monoglyceride is glyceryl monocaprylate.
(CC) The pharmaceutical composition of embodiment (BB), wherein the glyceryl monocaprylate comprises at least about 85 weight percent of the medium chain oil.
(DD) The pharmaceutical composition of embodiment (H), wherein the polyoxyethylene hydrogenated castor oil is a polyoxyl X hydrogenated castor oil and X is an integer from 1 to 100.
(EE) The pharmaceutical composition of embodiment (DD), wherein the polyoxyethylene hydrogenated castor oil is polyoxyl 40 hydrogenated castor oil.
(FF) The pharmaceutical composition of embodiment (EE), wherein the polyoxyl 40 hydrogenated castor oil comprises from about 1 to about 10 weight percent of the pharmaceutical composition.
(GG) The pharmaceutical composition of embodiment (FF), wherein the polyoxyl 40 hydrogenated castor oil comprises about 4 weight percent to about 5 weight percent of the pharmaceutical composition.
(HH) The pharmaceutical composition of any of embodiments (A) through (U), wherein the medium chain oil further comprises a diglyceride.
(II) The pharmaceutical composition of embodiment (HH), wherein the diglyceride is a mixed or complex diglyceride selected from the group consisting of glyceryl caproate/caprylate, glyceryl caproate/caprate, glyceryl caproate/laurate, glyceryl caproate/myristate, glyceryl caprylate/caprate, glyceryl caprylate/laurate, glyceryl caprylate/myristate, glyceryl caprate/laurate, glyceryl caprate/myristate, and glyceryl laurate/myristate.
(JJ) The pharmaceutical composition of embodiment (JJ), wherein the diglyceride is glyceryl caprylate/caprate.
(KK) The pharmaceutical composition of any of embodiments (HH) through (JJ), wherein the diglyceride comprises from about 5 weight percent to about 10 weight percent of the pharmaceutical composition.
(LL) The pharmaceutical composition of embodiment (KK), wherein the diglyceride comprises from about 8 to about 9 weight percent of the pharmaceutical composition.
(MM) The pharmaceutical composition of embodiments (I), wherein the d-α-tocopherol polyethylene glycol succinate derivative has the structure

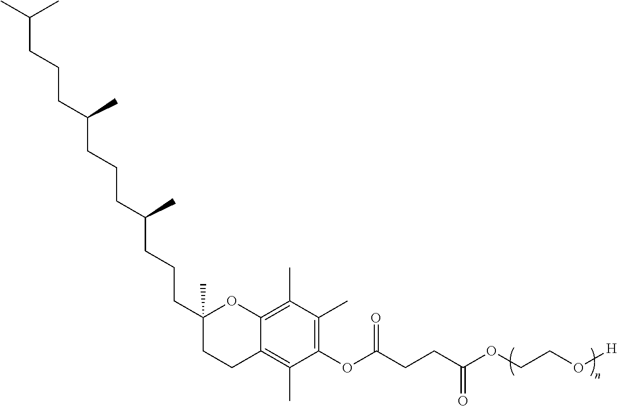

and n is 1-100.
(NN) The pharmaceutical composition of embodiment (MM), wherein n is 1 to 50.
(OO) The pharmaceutical composition of embodiment (NN), wherein n is 1 to 25.
(PP) The pharmaceutical composition of embodiment (OO), wherein n is about 22.
(QQ) The pharmaceutical composition of embodiment (MM), wherein the d-α-tocopherol polyethylene glycol succinate derivative is TPGS-1000.
(RR) The pharmaceutical compositions of any of embodiments (MM) through (QQ), wherein the d-α-tocopherol polyethylene glycol succinate derivative comprises from about 0.1 weight percent to about 5 weight percent of the pharmaceutical composition.
(SS) The pharmaceutical composition of embodiment (RR), wherein the d-α-tocopherol polyethylene glycol succinate derivative comprises from about 2 weight percent to about 3 weight percent of the pharmaceutical composition.
(TT) The pharmaceutical composition of embodiment (SS), wherein the d-α-tocopherol polyethylene glycol succinate derivative comprises about 2.3 weight percent or about 2.4 weight percent of the pharmaceutical composition.
(UU) The pharmaceutical composition of any of the foregoing embodiments, wherein the pharmaceutical composition provides an oral bioavailability for estradiol and/or progesterone of at least about 150 percent as measured by an increase in AUC when compared to a reference product, such as ESTRACE or PROMETRIUM.
(VV) The pharmaceutical composition of embodiment (UU), wherein the reference product is PROMETRIUM.
(WW) The pharmaceutical composition of any preceding embodiment, further comprising a terpene.
(XX) The pharmaceutical composition of embodiment (WW), wherein the terpene is d-limonene.
(YY) A method of treating a disease or condition associated with reduced estrogen levels and/or reduced progesterone levels, the method comprising administering to a subject in need thereof a pharmaceutical composition according to any of the preceding embodiments.
(ZZ) A method of treating a disease or condition associated with reduced estrogen levels and/or reduced progesterone levels, the method comprising administering to a subject in need thereof a pharmaceutical composition of any of the preceding embodiments.
(AAA) The method of embodiment (ZZ), wherein the disease or condition associated with reduced progesterone levels is selected from the group consisting of endometrial hyperplasia; secondary amenorrhea; prevention of preterm birth; and osteoporosis.
(BBB) The method of embodiment (AAA), the disease or condition associated with reduced progesterone levels is selected from the group consisting of hot flashes, hot flushes, night sweats, sleep disturbances, mood changes, vulvovaginal atrophy, or osteoporosis.
(CCC) A method of treating a disease or condition associated with reduced estradiol and/or reduced progesterone levels, the method comprising administering to a subject in need thereof a pharmaceutical composition comprising one or more predominantly medium chain oils, estradiol and progesterone, wherein the pharmaceutical composition has an AUC for estradiol that is at least 150% of the AUC of an equal amount of a reference product.
(DDD) The method of embodiment (CCC), wherein the pharmaceutical composition has a Cₘₐₓ for estradiol that is at least 150% of the Cₘₐₓ of an equal amount of a reference product.
(EEE) The pharmaceutical composition of any of the preceding embodiments, wherein estradiol and progesterone are the sole active agents.

### EXAMPLES

The pharmaceutical composition described herein is now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### Example 1: Pharmaceutical Compositions

Pharmaceutical compositions having both estradiol and progesterone as well as the ingredients shown in Tables 1-3 are readily prepared by combining the ingredients using standard preparatory techniques.

### Example 2: Pharmaceutical Compositions

Pharmaceutical compositions having the ingredients shown in Table 4 were prepared by combining the ingredients using standard preparatory techniques.

TABLE 4
Solubilized Progesterone Fill Formulas (all values presented in mg/g)

| | Pharma. Composition | | | | | | |
|---|---|---|---|---|---|---|---|
| Component | A | B | C | D | E | F | G |
| CAPMUL | 761.06 | 723.01 | 723.01 | 761.06 | 834.62 | — | 751.16 |
| 708G | | | | | | | |
| CAPMUL | 84.56 | 80.33 | 80.33 | 84.56 | — | 834.62 | 83.46 |
| MCM, NF | | | | | | | |
| Ultra High | — | 42.28 | 42.28 | — | — | — | — |
| Purity d- | | | | | | | |
| limonene | | | | | | | |
| BHT | 0.28 | 0.28 | 0.28 | 0.28 | — | — | — |
| Progesterone | 60.13 | 60.13 | 60.13 | 60.13 | 72.64 | 72.64 | 72.64 |
| Polysorbate 80 | 70.47 | 70.47 | 46.98 | 46.98 | 69.55 | 69.55 | 69.55 |
| TPGS 1000 | 23.49 | 23.49 | — | — | 23.18 | 23.18 | 23.18 |
| KOLLIPHOR | — | — | 46.98 | 46.98 | — | — | — |
| RH 40 | | | | | | | |

### Example 3: Particle Size Analysis

Average particle sizes for each of Pharmaceutical Compositions A, B, C, and D as disclosed in Example 2 were measured using a DELSA Nano photon correlation spectrometer. Approximately 0.5 g of a given sample was diluted with 55 mL of filtered deionized water and the mean size of the resulting particle and the zeta potential was calculated as set forth in Table 5. A similar particle size analysis can be performed for Pharmaceutical Compositions A1-U1 of Tables 1-3 of Example 1, which pharmaceutical compositions contain both estradiol and progesterone.

TABLE 5

| Pharmaceutical | Mean size (nm) ± | Zeta Potential |
|---|---|---|
| composition | Std. Dev. | (mV) |
| A | 301.6 ± 164.4 | −16.89 |
| B | 678.2 ± 698.6 | −16.87 |
| C | 575.2 ± 604.8 | −20.63 |
| D | 156.3 ± 52.2 | −18.37 |

### Example 4: Oral Bioavailability in Rats

Oral bioavailability of the pharmaceutical compositions was assessed in male Sprague-Dawley rats. According to the protocol, 30 male rats were divided into 6 groups of 5 rats each. The rats were then treated with one of the pharmaceutical compositions discussed in Example 2 (Compositions A, B, C, and D), Pharmaceutical Composition H (a non-micelle forming, fully-solubilized progesterone pharmaceutical composition within the scope of this disclosure described more fully in Table 6), or PROMETRIUM according to the schedule shown in Table 7.

TABLE 6
Pharmaceutical Composition H

| Component | Chemical Name | Quantity (mg/g) |
|---|---|---|
| CAPMUL | Glyceryl | 846 |
| 708G | Caprylate | |
| CAPMUL | Caprylic/capric | 94 |
| MCM, NF | mono/diglycerides | |
| Progesterone | API | 60 |

TABLE 7

| Study | |
|---|---|
| Day | Event |
| −4 | Animals were transferred to surgery facility and were |
| | group/gang housed. |
| −3 | Animals were observed. |
| −2 | Animals were observed. |
| −1 | Animals were fitted with jugular vein catheters (vaporized |
| | isoflurane anesthesia) and treated with analgesics. The animals |
| | were fasted for 12 hours starting at 8:00 PM. |
| 0 | Gavage capsules were filled with 20 μL of compound per |
| | capsule. Baseline plasma samples were collected, the animals |
| | received compound via capsule gavage, and additional plasma |
| | samples were taken at 10, 20, 40, 60, 90, 120, 180, and 240 |
| | minutes post dosing. Frozen plasma samples were shipped on |
| | dry ice for analysis. |

Although PROMETRIUM was dosed in a capsule filled with 20 μL of the PROMETRIUM formulation, the PROMETRIUM capsule contained at least 6 times as much progesterone (400 mg/g formulation) as the test pharmaceutical compositions (60 mg/g composition) due to the way in which PROMETRIUM is formulated.

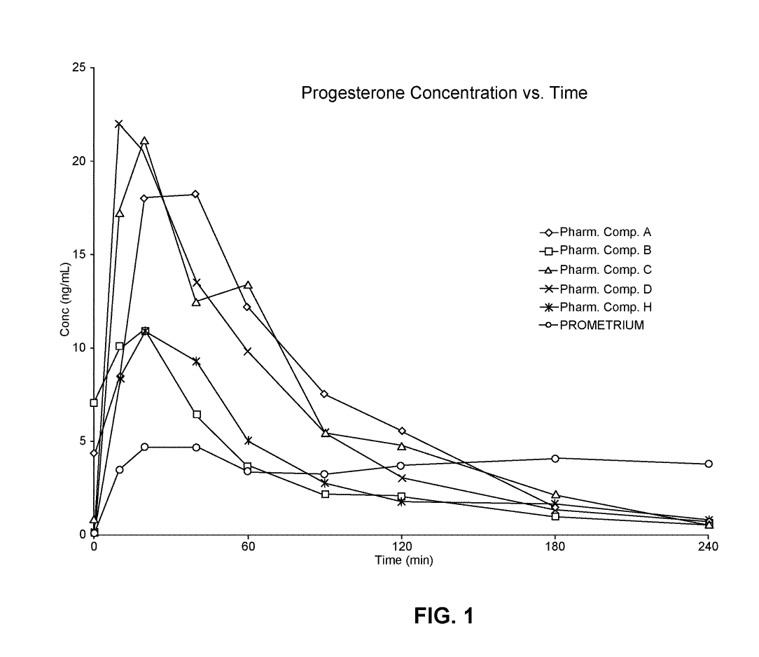

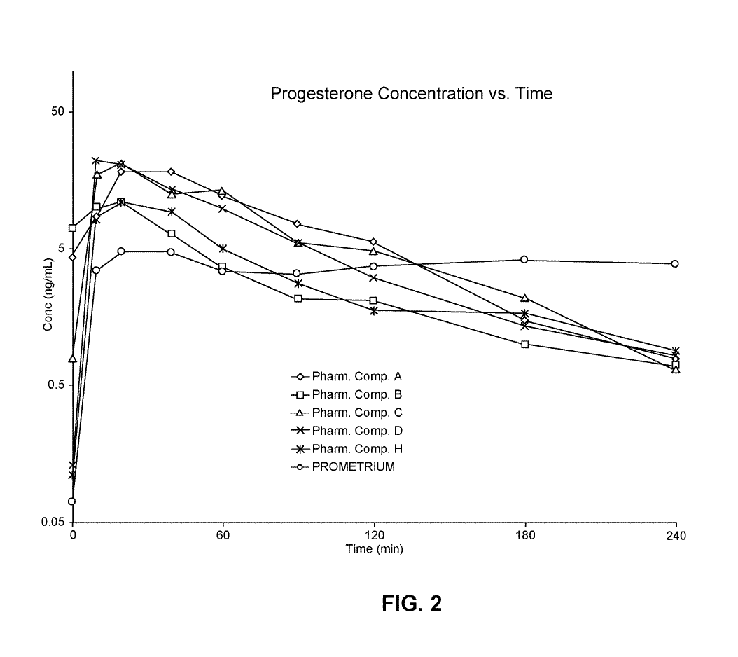

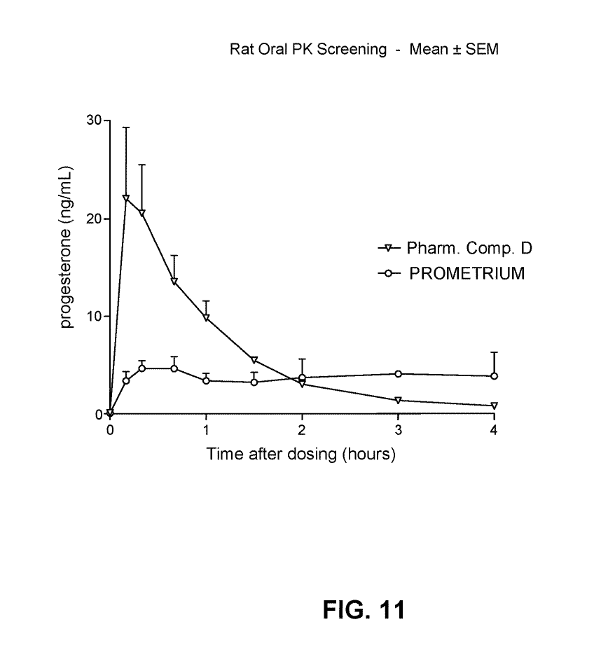

The frozen plasma samples were then analyzed and the data plotted. The results are shown in FIGS. 1 and 2 (linear-linear). Both figures show that test Pharmaceutical Compositions A, C, and D performed better than Pharmaceutical Composition H and PROMETRIUM. FIG. 11 shows the performance of Pharmaceutical Composition D and PROMETRIUM, as both shown in FIG. 1, in the absence of the other tested formulations.

The means of the PK parameters observed (+/−standard deviation) are shown in Table 8.

TABLE 8
Non-Normalized Progesterone PK Data

| | A | B | C | D | H | PROMETRIUM |
|---|---|---|---|---|---|---|
| Dose | 3.7 | 3.7 | 3.7 | 3.7 | 3.7 | 25 |
| (mg/kg) | | | | | | |
| Cₘₐₓ | 20.8 ± 12.8 | 13 ± 9 | 24.7 ± 11.2 | 23.5 ± 15.3 | 13.1 ± 6.9 | 6.9 ± 4.0 |
| (ng/mL) | | | | | | |
| tₘₐₓ (hr) | 0.467 ± 0.183 | 0.167 ± 0.167 | 0.4 ± 0.346 | 0.333 ± 0.204 | 0.367 ± 0.183 | 2.2 ± 1.609 |
| AUC₀₋ₜ | 27.3 ± 23.0 | 12.5 ± 9.2 | 26.2 ± 11.9 | 24.2 ± 8.8 | 14.1 ± 7.6 | 15.1 ± 8.4 |
| (ng-hr/mL) | | | | | | |
| AUC_{0-∞} | 28.0 ± 23.4 | 14.1 ± 11.1 | 27.1 ± 11.7 | 25.5 ± 8.2 | 15.6 ± 8.5 | 18.9 ± 13.6 |
| (ng-hr/mL) | | | | | | |

Despite containing significantly less progesterone than PROMETRIUM, each of the test pharmaceutical compositions provided a higher C. (greater than 10-fold) and AUC₀₋₄ (4.5 to 10-fold—except for Pharmaceutical Composition H, which showed an AUC similar to PROMETRIUM), when normalized to a standard 1 mg dose than was observed for PROMETRIUM. Each of the test pharmaceutical compositions had a higher Cₘₐₓ and shorter tₘₐₓ than PROMETRIUM, suggesting more rapid absorption.

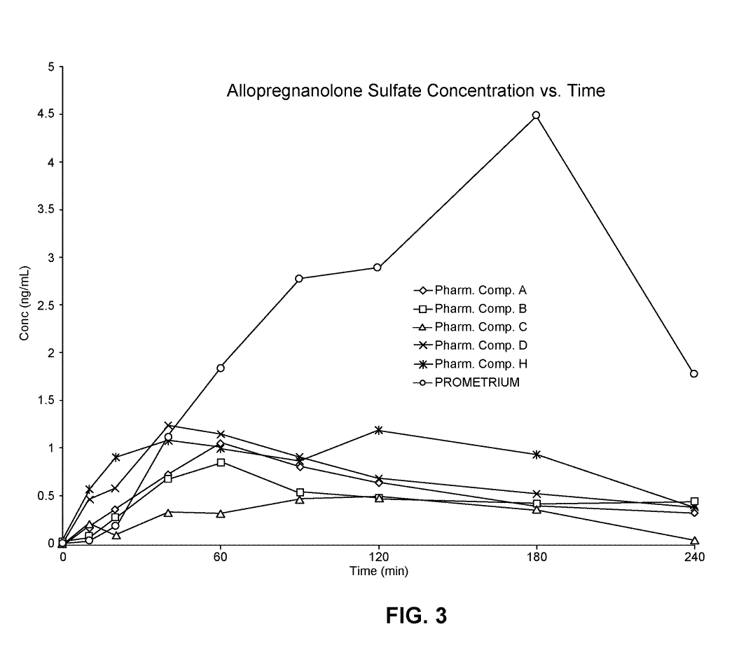

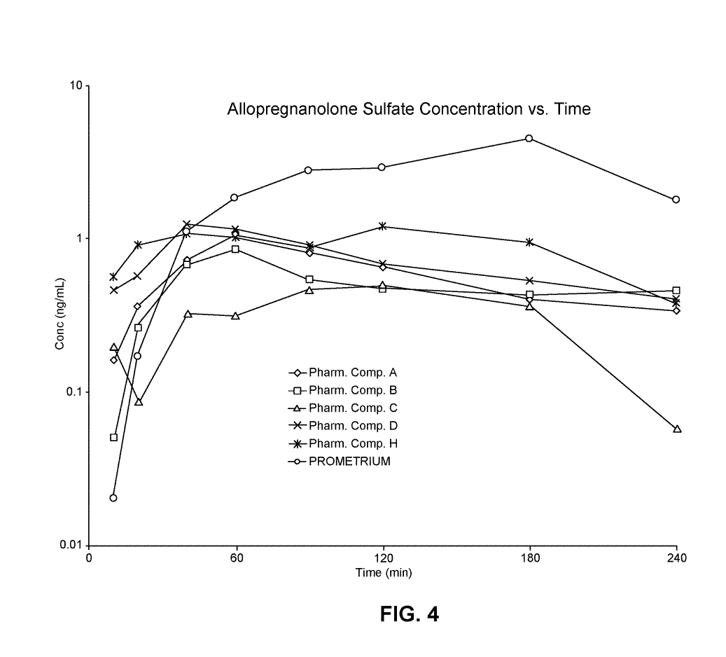

In addition, the relative amount of a down-stream metabolite of progesterone (allopregnanolone sulfate) was much higher in rats dosed with PROMETRIUM (AUC approximately 90% of the AUC for progesterone) than with the test pharmaceutical compositions (approximately 6-15%). Allopregnolone is believed to be associated with somnolence side effect in humans. In certain embodiments, Pharmaceutical Compositions A, B, C, or D can be administered to reduce or eliminate a somnolence side effect in patients needing progesterone therapy. See, FIGS. 3 and 4.

### Example 4: Food Effect on Oral Absorption

According to the protocol, 56 male Sprague-Dawley rats were divided into 8 groups of 7 rats each. Each group was given one of three test pharmaceutical compositions or PROMETRIUM as set forth in Table 9 according to the schedule shown in Table 10. Animals in “Fed” groups were presented with a pre-weighed amount of food 15 minutes prior to receiving a given pharmaceutical composition. The food was removed 45 minutes after dosing and weighed to calculate average consumption per animal. Animals in fasted groups received food approximately 4 hours after dosing.

TABLE 9

| | Pharmaceutical | |
|---|---|---|
| Group | Composition | Fed/Fasted |
| 1 | PROMETRIUM | Fasted |
| 2 | PROMETRIUM | Fed |
| 3 | D | Fasted |
| 4 | D | Fed |
| 5 | A | Fasted |
| 6 | A | Fed |
| 7 | C | Fasted |
| 8 | C | Fed |

TABLE 10

| Study | |
|---|---|
| Day | Event |
| −4 | Animals were transferred to surgery facility and were |
| | group/gang housed. |
| −3 | Animals were observed. |
| −2 | Animals were observed. |
| −1 | Animals were fitted with jugular vein catheters (vaporized |
| | isoflurane anesthesia) and treated with analgesics. The animals |
| | were fasted for 16 hours starting at 4:00 PM. |
| 0 | Gavage capsules were filled with 20 μL of composition per |
| | capsule. The Animals were either fed or fasted, as noted above, |
| | and given compositions via capsule gavage. Plasma samples |
| | were taken at 10, 20, 40, 60, 90, 120, 180, and 240 minutes |
| | post dosing. Frozen plasma samples were shipped on dry ice |
| | for analysis. |

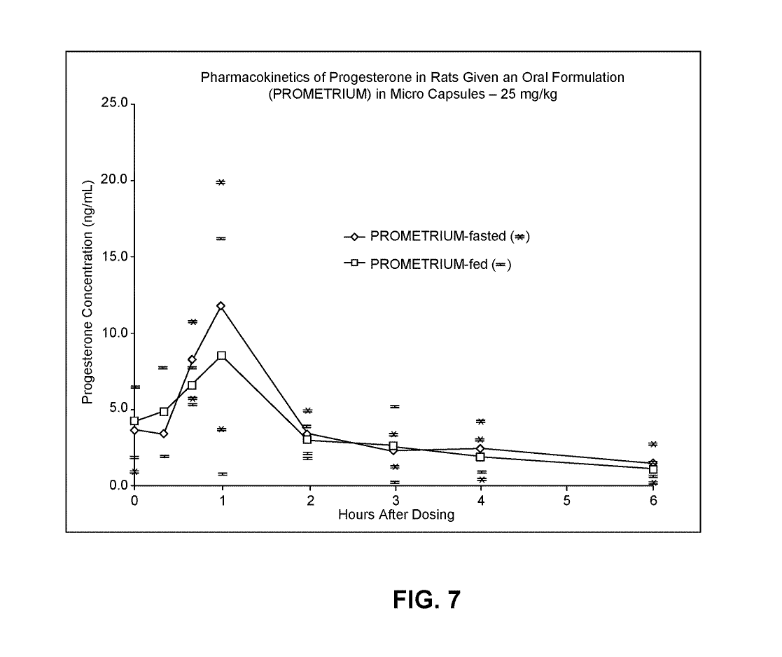

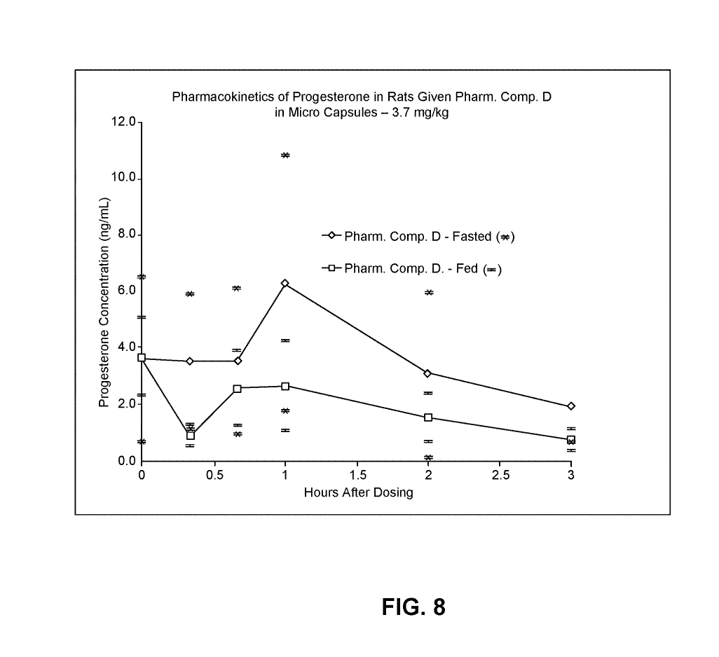

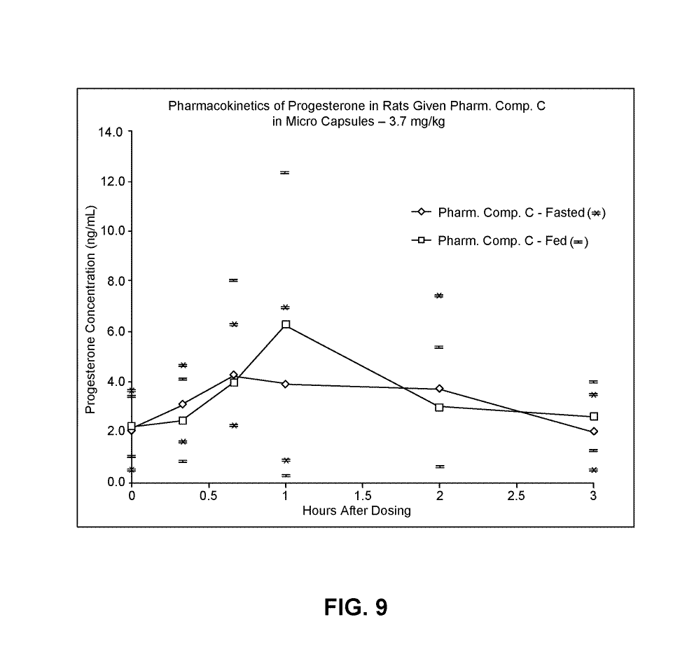

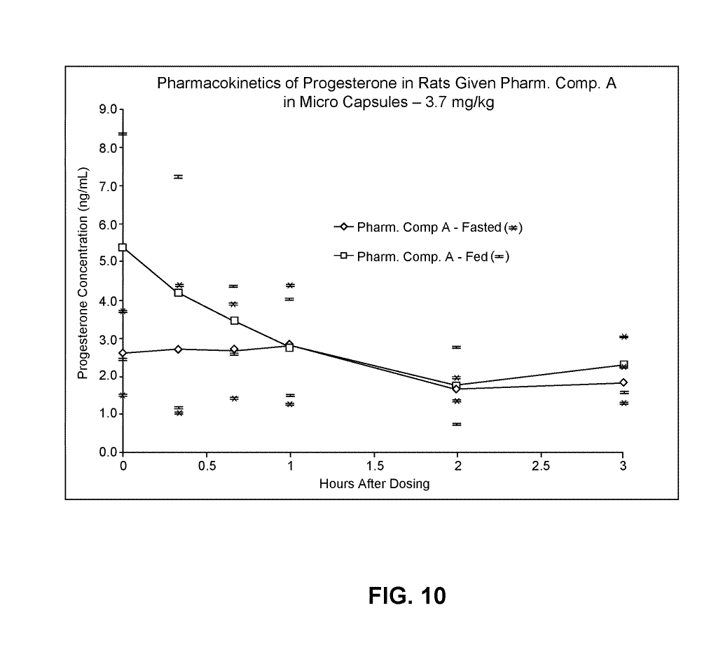

The results of this study are show in FIGS. 7, 8, 9, and 10 and show that there was no clear food effect on the PK of any of the pharmaceutical compositions, but dose-normalized progesterone exposure for the test pharmaceutical compositions was approximately 5-fold higher for Cₘₐₓ and 3-fold higher for AUCₗₐₛₜ than for PROMETRIUM.

In certain embodiments, the Cₘₐₓ and AUC₀₋ₜ differences between the test compositions and PROMETRIUM are surprising given that PROMETRIUM contains about 400 mg progesterone per gram of formulation, whereas the test pharmaceutical compositions contain 60 mg progesterone per gram of formulation (i.e. about 6 weight percent). In view of this significant difference in the amount of available progesterone when both compositions were dosed at equal volumes (i.e. 20 μl), a person of ordinary skill in the art would not have predicted that the present pharmaceutical composition would enhanced oral bioavailability versus the reference composition (PROMETRIUM).

The breadth and scope of the present invention should not be limited by any of the above-described exemplary embodiments, but should be defined only in accordance with the following claims and their equivalents.

All patents, patent applications, and other reference noted or referenced in this application are hereby incorporated by reference in their entirety.

1. A pharmaceutical composition for providing enhanced oral bioavailability of estradiol and/or progesterone, the pharmaceutical composition comprising:
estradiol, progesterone, a polysorbate, and a medium chain oil, wherein the medium chain oil comprises at least about 50 weight percent of a predominantly medium chain monoglyceride; and
the estradiol and the progesterone are both fully solubilized.

2. The pharmaceutical composition of claim 1, wherein the medium chain oil comprises at least about 55 weight percent of a predominantly medium chain monoglyceride.

3. The pharmaceutical composition of claim 1, wherein the medium chain oil comprises at least about 60 weight percent of a predominantly medium chain monoglyceride.

4. The pharmaceutical composition of claim 1, wherein the medium chain oil comprises at least about 65 weight percent of a predominantly medium chain monoglyceride.

5. The pharmaceutical composition of claim 1, wherein the medium chain oil comprises at least about 70 weight percent of a predominantly medium chain monoglyceride.

6. The pharmaceutical composition of claim 1, wherein the polysorbate is selected from the group consisting of polysorbate 20, polysorbate 40, polysorbate 60, polysorbate 65, and polysorbate 80.

7. The pharmaceutical composition of claim 1, wherein the pharmaceutical composition provides an oral bioavailability for estradiol and/or progesterone of at least about 150 percent as measured by an increase in AUC when compared to a reference product.

8. The pharmaceutical composition of claim 1, further comprising a polyoxyethylene hydrogenated castor oil.

9. The pharmaceutical composition of claim 1, further comprising a d-α-tocopherol polyethylene glycol succinate derivative.

10. The pharmaceutical composition of claim 1, further comprising a terpene.

11. The pharmaceutical composition of claim 1, wherein the predominantly medium chain monoglyceride, and the estradiol and progesterone are present at a weight ratio of about 10:1 to about 15:1.

12.-16. (canceled)

17. The pharmaceutical composition of claim 1, wherein the pharmaceutical composition provides an oral bioavailability for estradiol and/or progesterone of at least about 150 percent as measured by an increase in AUC when compared to a reference product.

18.-20. (canceled)

21. An oral, fully-solubilized estradiol and progesterone pharmaceutical composition comprising:
estradiol, progesterone and a polysorbate in a weight ratio of from about 1:2 to about 2:1; and
a medium chain oil comprising a mixture of medium chain mono- and diglycerides, the medium chain oil not containing more than about 10 weight percent triglycerides.

22.-26. (canceled)

27. A method of treating a disease or condition associated with reduced estrogen levels, the method comprising administering to a subject in need thereof a pharmaceutical composition according to claim 1.

28. (canceled)

29. The method of claim 27, wherein the disease or condition associated with reduced estrogen levels is selected from the group consisting of hot flushes/flashes, vaginal dryness, night sweats, urinary tract infections, loss of libido, heart disease, or osteoporosis.

30. (canceled)

31. The method of claim 27, wherein the disease or condition associated with reduced estrogen levels is a vasomotor symptom of menopause.

32. The method of claim 31, wherein the vasomotor symptom is selected from the group consisting of hot flashes, hot flushes, and night sweats.

33. A pharmaceutical composition for providing enhanced oral bioavailability of estradiol and/or progesterone, the pharmaceutical composition comprising:
estradiol, progesterone, a polysorbate, and a medium chain oil, wherein the medium chain oil comprises at least about 50 weight percent of a first medium chain oil component; and
the estradiol and progesterone are both fully solubilized.

34. The pharmaceutical composition of claim 33, wherein the medium chain oil further comprises a second medium chain oil component.

35. The pharmaceutical composition of claim 34, wherein the first medium chain oil component is a predominantly medium chain monoglyceride, and wherein the second medium chain oil component is a predominantly medium chain diglyceride.

36.-40. (canceled)

41. The pharmaceutical composition of claim 1, wherein the pharmaceutical composition forms micelles.

42.-44. (canceled)
